(19) United States
(12) Patent Application Publication
(10) Pub. No.: US 2024/0199516 A1
(43) Pub. Date: Jun. 20, 2024

(54) DIALKYLPENTENE ETHER AND DIALKYLPENTADIENE ETHER DERIVATIVES AS FRAGRANCES

(71) Applicant: BASF SE, Ludwigshafen am Rhein (DE)

(72) Inventors: Wolfgang KRAUSE, Lampertheim (DE); Ralf PELZER, Lampertheim (DE); Gunnar HEYDRICH, Ludwigshafen am Rhein (DE); Bernhard BRUNNER, Ludwigshafen am Rhein (DE)

(21) Appl. No.: 18/284,678

(22) PCT Filed: Mar. 29, 2022
(86) PCT No.: PCT/EP2022/058275

§ 371 Date: Sep. 28, 2023

(30) Foreign Application Priority Data
Apr. 1, 2021 (EP) 21166622.7

(51) Int. Cl. C07C 33/02; A61K 8/33; A61Q 13/00; C07C 33/20; C07C 43/15; C07C 43/166

(52) U.S. Cl. CPC C07C 33/02; A61K 8/33; A61Q 13/00; C07C 33/20; C07C 43/15; C07C 43/166; A61K 2800/77

(57) ABSTRACT

The present invention relates to dialkylpentene ether and dialkylpentadiene ether derivatives as fragrances. Specifically, it relates to the use of the ether derivatives as an aroma chemical and also for enhancing and/or modifying the aroma of a composition. The present invention is further directed to a composition comprising at least one dialkylpentene ether or dialkylpentadiene ether derivative and (i) at least one aroma chemical different from the dialkylpentene ether and dialkylpentadiene ether derivative or (ii) at least one non-aroma chemical carrier, or (iii) both (i) and (ii).

### FIELD OF INVENTION

The present invention relates to dialkylpentene ether and dialkylpentadiene ether derivatives as aroma chemicals to impart a fruity, sweet, technical, floral impression to a composition. Specifically, it relates to the use of the ether derivatives as an aroma chemical which imparts a fruity, sweet, technical or floral impression to a composition and also for enhancing and/or modifying the aroma of a composition. The present invention is further directed to a composition comprising at least one dialkylpentene ether or dialkylpentadiene ether derivative and (i) at least one aroma chemical different from the dialkylpentene ether and dialkylpentadiene ether derivative or (ii) at least one non-aroma chemical carrier, or (iii) both (i) and (ii).

### BACKGROUND

Aroma chemicals, especially fragrances, are of great interest, especially in the field of cosmetics, cleaning and laundry compositions. Fragrances of natural origin are mostly expensive, often limited in their available amount and, on account of fluctuations in environmental conditions, are also subject to variations in their content, purity etc. To circumvent these undesirable factors, it is therefore of great interest to create synthetic substances which have organoleptic properties that resemble more expensive natural fragrances, or which have novel and interesting sensory profiles.

Despite a large number of already existing synthetic aroma chemicals, there is a constant need for new components in order to be able to satisfy the multitude of properties desired for extremely diverse areas of application. These include, firstly, the sensory properties, i.e. the compounds should have advantageous odiferous (olfactory) properties. Furthermore, aroma chemicals should also have additional positive secondary properties, such as e.g. an efficient preparation method, the possibility of providing better sensory profiles as a result of synergistic effects with other aroma chemicals, a higher stability under certain application conditions, a higher extendibility, a better higher substantivity.

Such properties are of special interest for compositions such as for example body care compositions, hygiene articles, cleaning compositions, textile detergent compositions and compositions for scent dispensers.

Of special interest are aroma chemicals, which can impart one or more distinct sensory impressions to a composition, thereby contributing to a rich and interesting sensory profile, especially an olfactory profile of the composition. In this regard, aroma chemicals which can impart a fruity, spicy, sweet, floral, technical, animalic, musty and/or amber impression to a composition are of major interest. In addition, substantivity as well as tenacity are of special interest in order to obtain a long-lasting odiferous impression in the composition as well as on the surface which is treated with the composition.

However, since even small changes in chemical structure bring about massive changes in the sensory properties such as odor and/or flavor, the targeted search for substances with certain and distinct sensory properties such as a certain odor is extremely difficult. The search for new aroma chemicals is therefore in most cases difficult and laborious without knowing whether a substance with the desired odor and/or flavor will even actually be found.

It is an object of the presently claimed invention to provide substances which can be used as an aroma chemical either alone or as mixtures in compositions, in particular odor-intensive substances having a pleasant odor are sought. Furthermore, they should be combinable with other aroma chemicals, allowing the creation of novel advantageous sensory profiles and can be used in compositions.

It is an object of the presently claimed invention to provide new aroma chemicals which have pleasant olfactory impression; preferably a combination of the impression which are selected from the group consisting of fruity note, spicy note, sweet note, floral note, technical note, animalic note, musty note and amber note.

A further object of the present invention is that, the aroma chemicals should be obtainable from readily available starting materials, allowing their fast and economic manufacturing.

### SUMMARY OF THE INVENTION

This object is achieved by the provision of the inventive compounds which are Dialkylpentene ether or Dialkylpentadiene ether derivatives.

A first aspect of the presently claimed invention relates to a compound of formula (I)

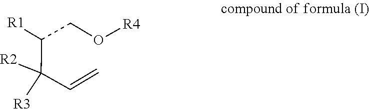

where the broken bond of compound of formula (I) is a single or double bond
wherein formula (I) comprises,
the compounds of formula (Ia)

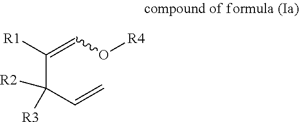

and the compounds of formula (Ib)

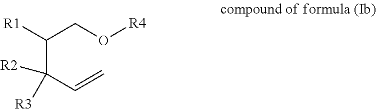

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
   or its salt or stereoisomer thereof.

In a further aspect the presently claimed invention relates to a compound of formula (Ib), wherein the formula (Ib) comprises the compounds of formula (Ib′)

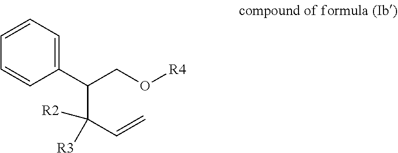

where
R₂ is linear or branched C₁-C₄ alkyl,
R₃ is linear or branched C₁-C₄ alkyl,
R₄ is H, linear or branched C₁-C₄ alkyl,
and the compounds of formula (Ib″)

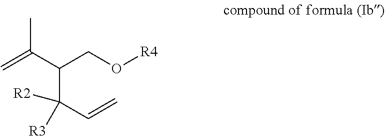

where
R₂ is linear or branched C₁-C₄ alkyl,
R₃ is linear or branched C₁-C₄ alkyl,
R₄ is linear or branched C₂-C₅ alkyl, linear or branched C₂-C₅ alkenyl,
   or its salts or stereoisomers thereof.

In another aspect, the presently claimed invention relates to a compound of formula (Ia)

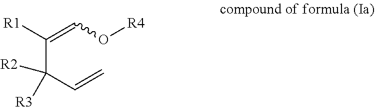

wherein R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
   or its salts or stereoisomers thereof.

In yet another aspect, the presently claimed invention relates to a compound of formula (Ib′)

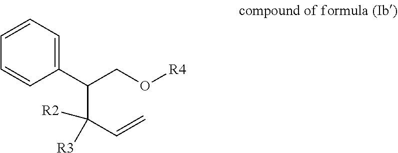

where
R₂ is linear or branched C₁-C₄ alkyl,
R₃ is linear or branched C₁-C₄ alkyl,
R₄ is H, linear or branched C₁-C₄ alkyl,
   or its salts or stereoisomers thereof.

In a further aspect, the presently claimed invention relates to a compound of formula (Ib″)

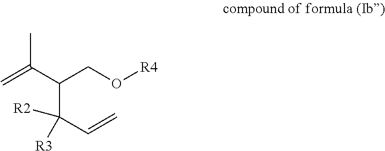

where
R₂ is linear or branched C₁-C₄ alkyl,
R₃ is linear or branched C₁-C₄ alkyl,
R₄ is linear or branched C₂-C₅ alkyl, linear or branched C₂-C₅ alkenyl,
   or its salts or stereoisomers thereof.

In another aspect the presently claimed invention relates to a compound of formula (I)

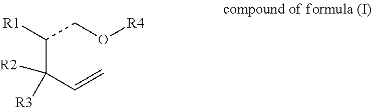

where the broken bond of compound of formula (I) is a single or double bond
wherein formula (I) comprises,
the compounds of formula (Ia)

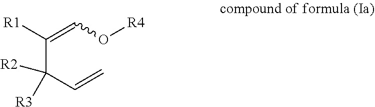

and the compounds of formula (Ib)

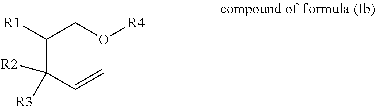

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
   wherein the compound of formula (Ib) comprises the compounds of formula (Ib′)

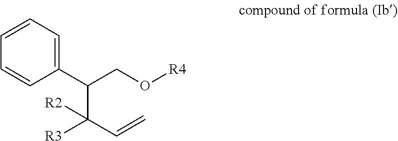

where
R₂ is linear or branched C₁-C₄ alkyl,
R₃ is linear or branched C₁-C₄ alkyl,
R₄ is H, linear or branched C₁-C₄ alkyl,
and the compounds of formula (Ib″)

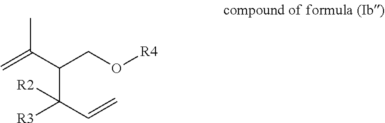

where
R₂ is linear or branched C₁-C₄ alkyl,
R₃ is linear or branched C₁-C₄ alkyl,
R₄ is linear or branched C₂-C₅ alkyl, linear or branched C₂-C₅ alkenyl,
   or its salts or stereoisomers thereof.

A yet another aspect of the presently claimed invention relates to the use of at least one compound of formula (I) wherein formula (I) comprises the compounds of formula (Ia) and the compounds of formula (Ib) as an aroma chemical, preferably as a fragrance.

A yet another aspect of the presently claimed invention relates to the use of at least one compound (Ia) as an aroma chemical, preferably as a fragrance.

A yet another aspect of the presently claimed invention relates to the use of at least one compound (Ib) as an aroma chemical, preferably as a fragrance.

A further aspect of the presently claimed invention relates to the use of at least one compound (Ib′) as an aroma chemical, preferably as a fragrance.

Another aspect of the presently claimed invention relates to the use of at least one compound (Ib″) as an aroma chemical, preferably as a fragrance.

A further aspect of the presently claimed invention relates to a method of imparting an aroma impression to a composition comprising at least the step of adding at least one compound of formula (I) to a composition.

A further aspect of the presently claimed invention relates to a method of imparting an aroma impression to a composition comprising at least the step of adding at least one compound (Ia) to a composition.

A further aspect of the presently claimed invention relates to a method of imparting an aroma impression to a composition comprising at least the step of adding at least one compound (Ib) to a composition.

A further aspect of the presently claimed invention relates to a method of imparting an aroma impression to a composition comprising at least the step of adding at least one compound (Ib′) to a composition.

A further aspect of the presently claimed invention relates to a method of imparting an aroma impression to a composition comprising at least the step of adding at least one compound (Ib″) to a composition.

A yet further aspect of the presently claimed invention relates to a composition comprising at least one compound (I) or at least one compound (Ia), or at least one compound (Ib), or at least one compound (Ib′) or at least one compound (Ib″) and
(i) at least one additional aroma chemical different from the compounds of the presently claimed invention, or
(ii) at least one non-aroma chemical carrier, or
(iii) a mixture of (i) and (ii).

Another aspect of the presently claimed invention relates to the use of at least one compound of formula (I) for modifying the aroma character of an aroma chemical composition.

A further aspect of the presently claimed invention relates to a method of boosting the aroma of a composition. Said method comprises the step of mixing at least one compound of formula (I) and mixtures thereof with other ingredients such as, e.g., at least one other aroma chemical different from the compound of formula (I) and/or at least one non-aroma chemical carrier so as to obtain the aroma chemical composition.

Yet another aspect of the presently claimed invention relates to a method of modifying the aroma of an aroma chemical composition. Said method comprises the step of incorporating at least one compound of formula (I) or mixtures thereof into an aroma chemical composition so as to obtain an aroma-modified composition.

Furthermore, the compounds of the presently claimed invention can be produced in good yields and purities by a simple synthesis starting from readily available starting materials. Thus, the compounds of the presently claimed invention can be produced in large scales and in a simple and cost-efficient manner.

### DETAILED DESCRIPTION

The following detailed description is merely exemplary in nature and is not intended to limit the presently claimed invention or the application and uses of the presently claimed invention. Furthermore, there is no intention to be bound by any theory presented in the preceding technical field, background, summary or the following detailed description.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the subject matter described herein are capable of operation in other sequences than described or illustrated herein. In case the terms “(A)”, “(B)” and “(C)” or AA), BB) and CC) or “(a)”, “(b)”, “(c)”, “(d)”, “(i)”, “(ii)” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” or “preferred embodiment” means that a particular feature, structure or characteristic described in connection with the embodiment is included in at least one embodiment of the presently claimed invention. Thus, appearances of the phrases “in one embodiment” or “In a preferred embodiment” or “in a preferred embodiment” in various places throughout this specification are not necessarily all referring to the same embodiment but may refer. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments are used in any combination.

Furthermore, the ranges defined throughout the specification include the end values as well, i.e. a range of 1 to 10 implies that both 1 and 10 are included in the range. For the avoidance of doubt, the applicant shall be entitled to any equivalents according to applicable law.

In the context of the present invention, the term “aroma” refers to a sensory property and comprises an odor and/or a flavor.

The term “aroma chemical” denotes a substance which is used to obtain a sensory or organoleptic (used interchangeably herein) impression and comprises its use to obtain an olfactory and/or a flavor impression. The term “olfactory impression” or “note” (used interchangeably here) denotes an odor impression without any positive or negative judgement, while the term “scent impression” or “fragrance impression” or “aroma impression” (used interchangeably herein) as used herein is connected to an odor impression which is generally felt as pleasant. Thus a “fragrance” or “scent” denotes an aroma chemical, which predominately induces a pleasant odor impression. A flavor denotes an aroma chemical, which induces a taste impression.

The term “aroma composition”, as used herein, refers to a composition which induces an aroma. The term aroma composition comprises “odor composition” and/or “flavor composition”. An odor composition being a composition, which predominately induces an odor impression, a flavor composition being a composition, which predominantly induces a taste impression.

The term “aroma profile” denotes the overall aroma impression of an aroma chemical and is composed of the individual aroma impressions of an aroma chemical.

The term odor composition comprises “fragrance composition” or “scent composition” (used interchangeably herein), which predominately induce an odor impression which is generally felt as pleasant.

The general hedonistic expressions “advantageous sensory properties” or “advantageous organoleptic properties” describe the niceness and conciseness of an organoleptic impression conveyed by an aroma chemical. “Niceness” and “conciseness” are terms which are familiar to the person skilled in the art, such as a perfumer. Niceness generally refers to a spontaneously brought about, positively perceived, pleasant sensory impression. However, “nice” does not have to be synonymous with “sweet”. “Nice” can also be the odor of musk or sandalwood. “Conciseness” generally refers to a spontaneously brought about sensory impression which—for the same test panel—brings about a reproducibly identical reminder of something specific. For example, a substance can have an odor which is spontaneously reminiscent of that of an “apple”: the odor would then be concisely of “apples”. If this apple odor were very pleasant because the odor is reminiscent, for example, of a sweet, fully ripe apple, the odor would be termed “nice”. However, the odor of a typically tart apple can also be concise. If both reactions arise upon smelling the substance, in the example thus a nice and concise apple odor, then this substance has particularly advantageous sensory properties.

The expressions “combination of”, “in combination with” or “combined with” when used herein referring to the compositions, methods or the use of two compounds, take account of the fact that the two compounds do not need to be used in the form of a physical mixture of said compounds but can be used (e.g., added) separately. Where the compounds are used separately, they can be used (e.g. added) sequentially (i.e. one after the other) in any order, or concurrently (i.e. basically at the same time).

The term “boosting”, or “boost” is used herein to describe the effect of enhancing and/or modifying the aroma of an aroma chemical or of a composition. The term “enhancing” comprises an improvement of the niceness and/or conciseness of an aroma and/or an improvement of the intensity. The term “modifying” comprises the change of an aroma profile. The terms “niceness” and “conciseness” are familiar to the person skilled in the art, such as a perfumer and have the respective meaning.

The intensity can be determined via a threshold value determination. A threshold value of an odor is the concentration of a substance in the relevant gas space at which an odor impression can just still be perceived by a representative test panel, although it no longer has to be defined.

Booster effects are particularly desired in fragrance composition when top-note-characterized applications are required, in which the odor is to be conveyed particularly quickly and intensively, for example in deodorants, air fresheners or in the taste sector in chewing gums.

The terms “the invention relates to” and “the invention is directed to” are used synonymously throughout the invention.

The term “tenacity” describes the evaporation behaviour over time of an aroma chemical. The tenacity can for example be determined by applying the aroma chemical to a test strip, and by subsequent olfactory evaluation of the odor impression of the test strip. For aroma chemicals with high tenacity the time span after which the panel can still identify an aroma impression is long.

The term “substantivity” describes the interaction of an aroma chemical with a surface, such as for example the skin or a textile, especially after subsequent treatment of the surface, such as for example washing. The substantivity can for example be determined by washing a textile with a textile detergent composition comprising the aroma chemical and subsequent olfactory evaluation of the textile directly after washing (wet textile) as well as evaluation of the dry textile after prolonged storage.

The term “stability” describes the behaviour of an aroma chemical upon contact with oxygen, light and/or other substances. An aroma chemical with high stability maintains its aroma profile over a long period in time, preferably in a large variety of compositions and under various storage conditions.

In order to impart a long-lasting aroma impression to a composition or to a surface treated with a composition, the tenacity, the substantivity as well as the stability of the aroma chemical in the compositions should preferably be high.

As used herein the terms “compound (I)” or “compounds (I)” refer to the compound(s) of formula (I) including, all sub-formula of formula (I), e.g. formula (Ia), formula (Ib), formula (Ib′) and (Ib″), all the stereoisomeric forms (stereoisomers) thereof in all ratios and the salts thereof. Preferably the terms “compound (I)” or “compounds (I)” refer to the compound(s) of formula (I) including all sub-formula of formula (I), e.g. formula (Ia), formula (Ib), formula (Ib′) and (Ib″) and all the stereoisomeric forms (stereoisomers) thereof in all ratios.

Likewise, this terminology applies to all sub-formula of formula (I), e.g. formula (Ia), formula (Ib), formula (Ib′) and (Ib″).

The term “stereoisomer” is a general term used for all isomers of individual compounds that differ only in the orientation of their atoms in space. The term stereoisomer includes mirror image isomers (enantiomers), mixtures of mirror image isomers (racemates, racemic mixtures), geometric (cis/trans or E/Z) isomers, and isomers of compounds with more than one chiral center that are not mirror images of one another (diastereoisomers).

The term “C₁-C₅-alkyl” refers to a straight-chained or branched saturated hydrocarbon group having 1 to 6 carbon atoms, for example, methyl, ethyl, propyl, 1-methylethyl, butyl, 1-methylpropyl, 2-methylpropyl, 1,1-dimethylethyl, pentyl, 1-methylbutyl, 2-methylbutyl, 3-methyl butyl, 2,2-dimethylpropyl, 1-ethylpropyl, 1,1-dimethylpropyl, 1,2-dimethylpropyl.

The term “C₂-C₅-alkenyl” refers to a straight-chain or branched unsaturated hydrocarbon radical having 2 to 5 carbon atoms and a double bond in any position. Examples are “C₂-C₅-alkenyl”groups, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

### Compound

One embodiment of the present invention is directed to at least one compound of formula (I)

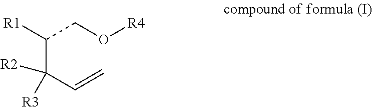

where the broken bond of compound of formula (I) is a single or double bond
wherein formula (I) comprises,
the compounds of formula (Ia)

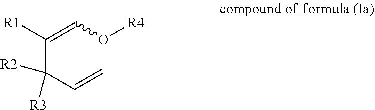

and the compounds of formula (Ib)

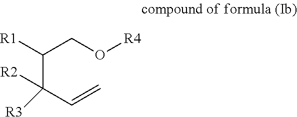

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
   or its salt or stereoisomer thereof.

A preferred embodiment of the present invention is directed to a compound of formula (Ib), wherein the formula (Ib) comprises the compounds of formula (Ib′)

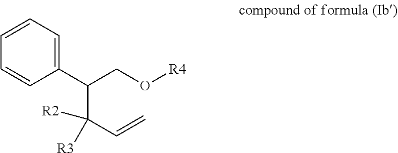

where
R₂ is linear or branched C₁-C₄ alkyl,
R₃ is linear or branched C₁-C₄ alkyl,
R₄ is H, linear or branched C₁-C₄ alkyl,
and the compounds of formula (Ib″)

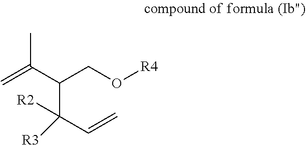

where
R₂ is linear or branched C₁-C₄ alkyl,
R₃ is linear or branched C₁-C₄ alkyl,
R₄ is linear or branched C₂-C₅ alkyl, linear or branched C₂-C₅ alkenyl,
   or its salt or stereoisomer thereof.

Another preferred embodiment of the present invention is directed to at least one compound of formula (Ia)

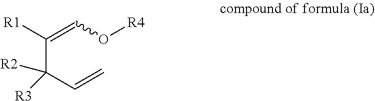

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
   or its salt or stereoisomer thereof.

In another preferred embodiment, the at least one compound of formula (Ia) is selected from a group consisting of compounds wherein
R₁ is linear or branched C₂-C₄ alkenyl,
R₂ is methyl,
R₃ is methyl,
R₄ is linear C₁-C₃ alkyl, branched C₅ alkenyl.

Another preferred embodiment of the present invention is directed to a compound of formula (Ib′)

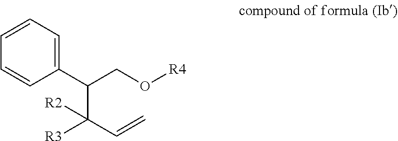

where
R₂ is linear or branched C₁-C₄ alkyl,
R₃ is linear or branched C₁-C₄ alkyl,
R₄ is H, linear or branched C₁-C₄ alkyl,
   or its salt or stereoisomer thereof.

In another preferred embodiment, the at least one compound of formula (Ib′) is selected from a group consisting of compounds wherein
R₂ is methyl,
R₃ is methyl,
R₄ is H, linear C₁-C₃ alkyl.

Another preferred embodiment of the present invention is directed to a compound of formula (Ib″)

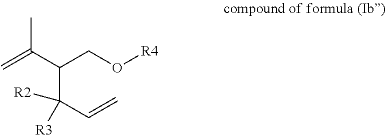

where
R₂ is linear or branched C₁-C₄ alkyl,
R₃ is linear or branched C₁-C₄ alkyl,
R₄ is linear or branched C₂-C₅ alkyl or linear or branched C₂-C₅ alkenyl,
   or its salt or stereoisomer thereof.

In preferred embodiment, the at least one compound of formula (Ib″) is selected from a group consisting of compounds wherein
R₂ is methyl,
R₃ is methyl,
R₄ is C₅ alkenyl or linear C₂-C₃ alkyl.

A further preferred embodiment of the present invention is directed to the compound of formula (I)

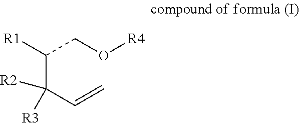

where the broken bond of compound of formula (I) is a single or double bond wherein formula (I) comprises,
the compounds of formula (Ia)

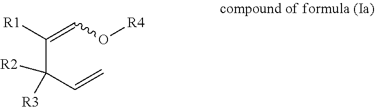

and compounds of formula (Ib)

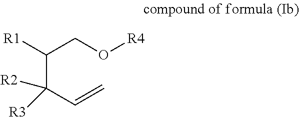

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
wherein, the formula (Ib) comprises at least one compounds of formula (Ib′)

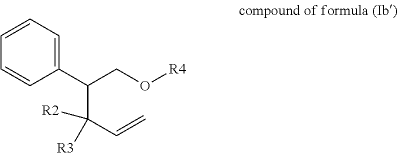

where
R₂ is linear or branched C₁-C₄ alkyl,
R₃ is linear or branched C₁-C₄ alkyl,
R₄ is H, linear or branched C₁-C₄ alkyl,
and at least one compound of formula (Ib″)

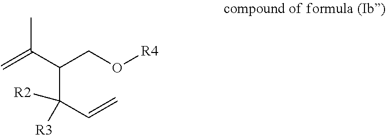

where
R₂ is linear or branched C₁-C₄ alkyl,
R₃ is linear or branched C₁-C₄ alkyl,
R₄ is linear or branched C₂-C₅ alkyl or linear or branched C₂-C₅ alkenyl.
or its salt or stereoisomer thereof.

### Use

### The Presently Claimed Invention is Directed to the Use of at Least One Compound of Formula (I)

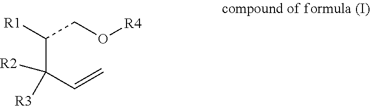 [0177] where the broken bond of compound of formula (I) is a single or double bond
wherein formula (I) comprises,
the compounds of formula (Ia)

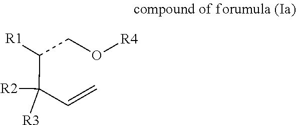

and the compounds of formula (Ib)

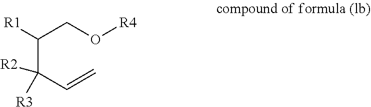

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
   or its salt or stereoisomer thereof,
   as an aroma chemical or to impart an aroma impression to a composition.

A further preferred embodiment of the present invention is directed to the use of the compound of formula (I)

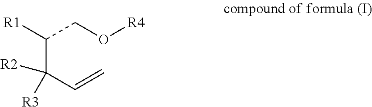

where the broken bond of compound of formula (I) is a single or double bond wherein formula (I) comprises,
the compounds of formula (Ia)

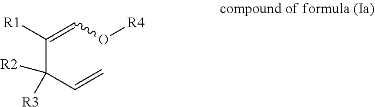

and compounds of formula (Ib)

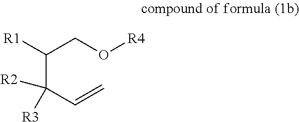

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
wherein, the formula (Ib) comprises at least one compounds of formula (Ib′)

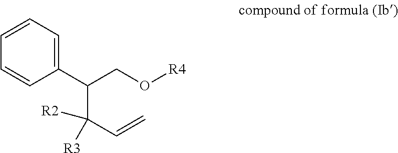

where
R₂ is linear or branched C₁-C₄ alkyl,
R₃ is linear or branched C₁-C₄ alkyl,
R₄ is H, linear or branched C₁-C₄ alkyl,
and at least one compound of formula (Ib″)

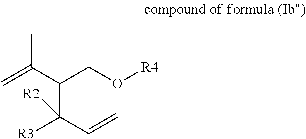

where
R₂ is linear or branched C₁-C₄ alkyl,
R₃ is linear or branched C₁-C₄ alkyl,
R₄ is linear or branched C₁-C₅ alkyl, linear or branched C₂-C₅ alkenyl,
or its salt or stereoisomer thereof,
   as an aroma chemical or to impart an aroma impression to a composition.

A further embodiment of the presently claimed invention is directed to the use of at least one compound of formula (I) to impart an aroma impression to a composition.

Another embodiment of the presently claimed invention is directed to the use of at least one compound of formula (I), as an aroma chemical.

In a further embodiment of the presently claimed invention is directed to the use of at least one compound of formula (I) to boost the aroma of a composition or as a frangrance or fragrance composition.

A preferred embodiment of the presently claimed invention is direct to the use of at least one compound of formula (Ia) as aroma chemical to impart at least one or more notes selected from an animalic note, a floral note and a musty note to a composition.

Another preferred embodiment of the presently claimed invention is directed to the use of at least one compound of formula (Ib′) as aroma chemical to impart at least one or more notes selected from an amber note, a sweet note and a fruity note to a composition.

Another further embodiment of the presently claimed invention is directed to the use of at least one compound of formula (Ib″) as aroma chemical to impart at least one or more notes selected from a technical note, a fruity note and a spicy note to a composition.

A further embodiment of the presently claimed invention is directed to the use of at least one compound (I), or at least one compound (Ia), or at least one compound (Ib), or at least one compound (Ib′) or at least one compound (Ib″), to boost the aroma of a composition.

In an embodiment of the presently claimed invention, at least one compound (I) or at least one compound (Ia), or at least one compound (Ib), or at least one compound (Ib′) or at least one compound (Ib″) is used as a fragrance or fragrance composition.

Preferably, at least one compound of formula (I), is used in a composition selected from the group consisting of perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

The compounds according to the presently claimed invention are used as aroma chemicals/in aroma compositions, preferably as a fragrance, in the above compositions.

### Composition

In an embodiment, the presently claimed invention relates to a composition comprising at least one compound (I):
(i) at least one aroma chemical (X) other than at least one compound (I) or
(ii) at least one non-aroma chemical carrier, or
(iii) both of (i) and (ii).

In a preferred embodiment, the presently claimed invention relates to a composition comprising at least one compound (I), wherein the total amount of compound (I), is in the range of ≥0.01 wt. % to ≤70.0 wt. %, based on the total weight of the composition.

In another preferred embodiment, the presently claimed invention relates to a composition comprising at least one compound (I), wherein the total amount of compounds (I), is in the range of ≥0.1 wt. % to ≤70.0 wt. %, based on the total weight of the composition.

In another preferred embodiment, the presently claimed invention relates to a composition comprising at least one compound (Ia)
(i) at least one aroma chemical (X) other than compound (Ia) or
(ii) at least one non-aroma chemical carrier, or
(iii) both of (i) and (ii).

In another preferred embodiment, the presently claimed invention relates to a composition comprising at least one compound (Ib):
(i) at least one aroma chemical (X) other than compound (Ib) or
(ii) at least one non-aroma chemical carrier, or
(iii) both of (i) and (ii).

In another embodiment, the presently claimed invention relates to a composition comprising at least one compound (Ib′),
(i) at least one aroma chemical (X) other than compound (Ib′) or
(ii) at least one non-aroma chemical carrier, or
(iii) both of (i) and (ii).

In another preferred embodiment, the presently claimed invention relates to a composition comprising at least one compound (Ib″),
(i) at least one one aroma chemical (X) other than compound (Ib″) or
(ii) at least one non-aroma chemical carrier, or
(iii) both of (i) and (ii).

Preferably in any of the above embodiment, the composition comprising at least one compound (I), or at least one compound (Ia), or at least one compound (Ib), or at least one compound (Ib′) or at least one compound (Ib″) wherein the total amount of compound (I) or (Ia) or (Ib) or (Ib′) or (Ib″) respectively is in the range of ≥0.01 wt. % to ≤70.0 wt. %, based on the total weight of the composition.

Further preferably in any of the above embodiment, the composition comprising at least one compound (I), or at least one compound (Ia), or at least one compound (Ib), or at least one compound (Ib′) or at least one compound (Ib″) wherein the total amount of compound (I) or (Ia) or (Ib) or (Ib′) or (Ib″) respectively is in the range of ≥0.1 wt. % to ≤70.0 wt. %, based on the total weight of the composition.

Preferably, the composition is an aroma composition preferably a fragrance composition.

Aroma chemical (X) different from the compounds of the present invention.

In one embodiment, the mixture comprises one aroma chemical which is different from the compounds of formula (I). Aroma chemicals which are different from the compounds of formula (I) are also referred to as aroma chemical (X).

The aroma chemical (X) is different from the compounds according to the invention.

The aroma chemical (X) is preferably selected from the group consisting of: geranyl acetate, alpha-hexylcinnamaldehyde, 2-phenoxyethyl isobutyrate, dihydromyrcenol, methyl dihydrojasmonate, 4,6,6,7,8,8-hexamethyl-1,3,4,6,7,8-hexahydro-cyclopenta[g]benzopyran, tetrahydrolinalool, ethyllinalool, benzyl salicylate, 2-methyl-3-(4-tert-butylphenyl)propanal, cinnamyl alcohol, 4,7-methano-3a,4,5,6,7,7a-hexahydro-5-indenyl acetate and/or 4,7-methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone and/or 2-acetyl-1,2,3,4,6,7,8-octahydro-2,3,8,8-tetramethylnaphthalene, hexyl salicylate, 4-tert-butylcyclohexyl acetate, 2-tert-butylcyclohexyl acetate, alpha-ionone, n-alpha-methylionone, alpha-isomethylionone, coumarin, terpinyl acetate, 2-phenylethyl alcohol, 4-(4-hydroxy-4-methylpentyl)-3-cyclohexene-carboxaldehyde, alpha-amylcinnamaldehyde, ethylene brassylate, (E)- and/or (Z)-3-methylcyclopentadec-5-enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3-(3-isopropylphenyl)butanal, 2-methyl-3-(3,4-methylenedioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate, 2,5,5-trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl) acetate and 3-[5,5,6-trimethylbicyclo[2.2.1]hept-2-yl]cyclohexan-1-ol.

In yet another preferred embodiment, the at least one aroma chemical (i) is selected from the group consisting of methyl benzoate, benzyl acetate, geranyl acetate, 2-isobutyl-4-methyltetrahydro-2H-pyran-4-ol, linalool, 2-isobutyl-4-methyltetrahydro-2H-pyran-4-ol and methyl benzoate.

In yet another preferred embodiment, the at least one aroma chemical (i) is selected from the group consisting of ethylvanillin, vanillin, 2,5-dimethyl-4-hydroxy-2H-furan-3-one (furaneol) or 3-hydroxy-2-methyl-4H-pyran-4-one (maltol).

Further aroma chemicals with which the compound of the present invention can be combined to give a composition according to the presently claimed invention can be found, e.g., in S. Arctander, Perfume and Flavor Chemicals, Vol. I and II, Montclair, N. J., 1969, self-published or K. Bauer, D. Garbe and H. Surburg, Common Fragrance and Flavor Materials, 4th Ed., Wiley-VCH, Weinheim 2001. Specifically, mention may be made of:
extracts from natural raw materials such as essential oils, concretes, absolutes, resins, resinoids, balsams, tinctures such as e.g.
ambergris tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; hyssop oil; civet absolute; cinnamon leaf oil; cinnamon bark oil, and fractions thereof, or ingredients isolated therefrom;
individual fragrances from the group of hydrocarbons, such as e.g. 3 carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1 octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10 trimethyl-5,9-undecadienal; heptanal diethylacetal; 1,1-dimethoxy-2,2,5 trimethyl-4-hexene; citronellyloxyacetaldehyde; (E/Z)-1-(1-methoxypropoxy)-hex-3-ene; the aliphatic ketones and oximes thereof such as e.g. 2-heptanone; 2-octanone; 3-octanone; 2-nonanone; 5-methyl-3-heptanone; 5-methyl-3 heptanone oxime; 2,4,4,7-tetramethyl-6-octen-3-one; 6-methyl-5-hepten-2-one;
the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles such as e.g. 2-nonenenitrile; 2-undecenenitrile; 2 tridecenenitrile; 3,12-tridecadienenitrile; 3,7-dimethyl-2,6-octadienenitrile; 3,7-dimethyl-6 octenenitrile;
the esters of aliphatic carboxylic acids such as e.g. (E) and (Z)-3-hexenyl formate; ethyl acetoacetate; isoamyl acetate; hexyl acetate; 3,5,5-trimethylhexyl acetate; 3 methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E) and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl-3,7-dimethyl-2,6-octadienoate; 4-methyl-2-pentyl crotonate;
the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2 ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2 butenoates thereof;
the acyclic terpene aldehydes and ketones such as e.g. geranial; neral; citronellal; 7 hydroxy-3,7-dimethyloctanal; 7 methoxy-3,7-dimethyloctanal; 2,6,10-trimethyl-9 undecenal; geranyl acetone; as well as the dimethyl and diethylacetals of geranial, neral, 7-hydroxy-3,7-dimethyloctanal; the cyclic terpene alcohols such as e.g. menthol; isopulegol; alpha-terpineol; terpine-4-ol; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones such as e.g. menthone; isomenthone; 8 mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methano-naphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as e.g. alpha-3,3-trimethylcyclohexylmethanol; 1 (4-isopropylcyclohexyl)ethanol; 2-methyl-4-(2,2,3-trimethyl-3-cyclopent-1-yl)butanol; 2-methyl-4-(2,2,3 trimethyl-3-cyclopent-1-yl)-2-buten-1-ol; 2-ethyl-4-(2,2,3-trimethyl-3 cyclopent-1-yl)-2-buten-1-ol; 3-methyl-5-(2,2,3 trimethyl-3-cyclopent-1-yl)pentan-2 ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers such as e.g. cineol; cedryl methyl ether; cyclododecyl methyl ether; 1,1-dimethoxycyclododecane; (ethoxymethoxy)cyclo-dodecane; alpha-cedrene epoxide; 3a,6,6,9a-tetramethyldodecahydronaphtho[2,1-b]furan; 3a-ethyl-6,6,9a-trimethyldodecahydro-naphtho[2, 1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones such as e.g. 4-tert-butylcyclohexanone; 2,2,5 trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2 cyclopenten-1-one; 3-methyl-2-pentyl-2-cyclopenten-1-one; 3-methyl-4-cyclopenta-decenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclo-hexadecen-1-one; 7-(7/8)-cyclohexadecen-1-one; 9 cyclo-heptadecen-1-one; cyclohexadecen-1-one; cyclopentadecanone; cyclohexadecanone;
the cycloaliphatic aldehydes such as e.g. 2,4-dimethyl-3-cyclohexenecarbaldehyde; 2 methyl-4-(2,2,6-trimethylcyclohexen-1-yl)-2-butenal; 4-(4-hydroxy-4-methylpentyl)-3 cyclohexene carbaldehyde; 4-(4-methyl-3-penten-1-yl)-3-cyclohexenecarbaldehyde;
the cycloaliphatic ketones such as e.g. 1-(3,3-dimethylcyclohexyl)-4-penten-1-one; 2,2 dimethyl-1-(2,4-dimethyl-3-cyclohexen-1-yl)-1-propanone; 1-(5,5-dimethyl-1 cyclo-hexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7 methanooctahydro-5 or 6-indenyl acetate;
the esters of cycloaliphatic alcohols such as e.g. 1-cyclohexylethyl crotonate;
the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6 dimethyl-2-cyclohexenecarboxylate; ethyl 2,3,6,6-tetramethyl-2 cyclohexene-carboxylate; ethyl 2-methyl-1,3-dioxolane-2-acetate;
the araliphatic alcohols such as e.g. benzyl alcohol; 1-phenylethyl alcohol, 2 phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2 phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxy-benzyl alcohol; 1-(4-isopropylphenyl)ethanol;
the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2 phenylethyl isovalerate; 1 phenylethyl acetate; alpha-trichloromethylbenzyl acetate; alpha,alpha-dimethylphenylethyl acetate; alpha, alpha-dimethylphenylethyl butyrate; cinnamyl acetate; 2-phenoxyethyl isobutyrate; 4-methoxybenzyl acetate;
the araliphatic ethers such as e.g. 2-phenylethyl methyl ether; 2 phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m dioxine;
the aromatic and araliphatic aldehydes such as e.g. benzaldehyde; phenylacetaldehyde; 3-phenylpropanal; hydratropaaldehyde; 4-methylbenzaldehyde; 4 methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3 methyl-5-phenylpentanal; 4-methoxybenzaldehyde; 4-hydroxy-3 methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as e.g. acetophenone; 4-methylacetophenone; 4-methoxyacetophenone; 4-tert-butyl-2,6-dimethylaceto-phenone; 4-phenyl-2-butanone; 4-(4-hydroxyphenyl)-2-butanone; 1-(2-naphthalenyl)-ethanone; 2-benzofuranylethanone; (3-methyl-2-benzofuranyl)ethanone; benzo¬phenone; 1,1,2,3,3,6-hexamethyl-5-indanyl methyl ketone; 6-tert-butyl-1,1 dimethyl-4 indanyl methyl ketone; 1-[2,3-dihydro-1,1,2,6-tetramethyl-3-(1-methylethyl)-1H-5 indenyl]ethanone; 5′,6′,7′,8′-tetrahydro-3′,5′,5′,6′,8′,8′-hexamethyl-2-acetonaphthone;
the aromatic and aliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as e.g. 2,4,6-trinitro-1,3-dimethyl-5 tert-butylbenzene; 3,5-dinitro-2,6-dimethyl-4-tert-butylacetophenone; cinnamonitrile; 3 methyl-5-phenyl-2-pentenonitrile; 3-methyl-5-phenylpentanonitrile; methyl anthranilate; methyl-N-methylanthranilate; Schiff bases of methyl anthranilate with 7 hydroxy-3,7-dimethyloctanal, 2-methyl-3-(4-tert-butylphenyl)propanal or 2,4 dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3 isopropyl-pyrazine; 2-isobutyl-3-methoxypyrazine;
the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2 ethoxy-5-(1-propenyl)phenol; p-cresyl phenylacetate;
the heterocyclic compounds such as e.g. 2,5-dimethyl-4-hydroxy-2H-furan-3-one; 2 ethyl-4-hydroxy-5-methyl-2H-furan-3-one; 3-hydroxy-2-methyl-4H-pyran-4-one; 2 ethyl-3-hydroxy-4H-pyran-4-one;
the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In a preferred embodiment, the composition comprises at least one compound of formula (I) and at least one aroma chemical (X).

The aroma chemical (X) used in the composition are obtained from known commercial sources and procured in Germany.

### Non-Aroma Chemical Carrier

The non-aroma chemical carrier in the composition of the invention is preferably selected from the group consisting of surfactants, oil components antioxidants, deodorant-active agents and solvents.

Preferably the at least one non-aroma chemical carrier is a compound, a mixture of compounds or other additives, which has/have no or no noteworthy sensory properties. The non-aroma chemical carrier can serve for the dilution and/or the fixing of the compounds of formula (I) and—optionally the at least one aroma chemical (X), as defined above, if comprised in the composition.

The non-aroma chemical carrier in the composition of the invention is preferably selected from the group consisting of surfactants, oil components, solvents or any mixture of two or more of the aforementioned.

### Solvent

In the context of the presently claimed invention, a “solvent” serves for the dilution of the compound of the present invention to be used according to the invention and/or any further component of the composition without having its own aroma.

The amount of solvent(s) is selected depending on the composition.

In yet another preferred embodiment, the solvent is present in the composition in a total amount of 0.01 wt. % to 99.0 wt. %, more preferably in a total amount of 0.05 wt. % to 95.0 wt. %, yet more preferably in a total amount of 0.1 wt. % to 80.0 wt. %, most preferably 0.1 wt. % to 70.0 wt. %, particularly in a total amount of 0.1 wt. % to 60.0 wt. %, based on the total weight of the composition.

In yet another preferred embodiment of the invention, the composition comprises 0.05 wt. % to 10 wt. %, more preferably 0.1 wt. % to 5 wt. %, yet more preferably 0.2 wt. % to 3 wt. % total solvent(s), based on the total weight of the composition. In yet another preferred embodiment of the invention, the composition comprises 20 wt. % to 70 wt. %, more preferably 25 wt. % to 50 wt. % of total solvent(s), based on the total weight of the composition.

In yet another preferred embodiment, the solvent is selected from the group consisting of ethanol, isopropanol, diethylene glycol monoethyl ether, glycerol, propylene glycol, 1,2-butylene glycol, dipropylene glycol, triethyl citrate, isopropyl myristate and any mixture of two or more of the aforementioned.

In a preferred embodiment, the composition comprises at least one compound of formula (I) and at least one solvent and optionally at least one aroma chemical (X).

In a preferred embodiment, the composition comprises at least one compound (I), or at least one compound (Ia), or at least one compound (Ib), or at least one compound (Ib′) or at least one compound (Ib″) and at least one solvent and optionally at least one aroma chemical (X).

### Oil Component

Preferably, the total oil components are present in an amount of 0.1 to 80 wt. %, more preferably 0.5 to 70 wt. %, yet more preferably 1 to 60 wt. %, even more preferably 1 to 50 wt. %, particularly 1 to 40 wt. %, more particularly 5 to 25 wt. % and specifically 5 to 15 wt. %, based on the total weight of the composition.

Preferably, the oil components may be selected, for example, from Guerbet alcohols based on fatty alcohols containing 6 to 18, preferably 8 to 10, carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C18-C38 alkyl-hydroxycarboxylic acids with linear or branched C6-C22 fatty alcohols, more especially dioctyl malate, esters of linear and/or branched fatty acids with polyhydric alcohols (for example propylene glycol, dimer dial or trimer triol), triglycerides based on C6-C10 fatty acids, liquid mono-, di- and triglyceride mixtures based on C6-C18 fatty acids, esters of C6-C22 fatty alcohols and/or Guerbet alcohols with aromatic carboxylic acids, more particularly benzoic acid, esters of dicarboxylic acids with polyols containing 2 to 10 carbon atoms and 2 to 6 hydroxyl groups, vegetable oils, branched primary alcohols, substituted cyclohexanes, linear and branched C6-C22 fatty alcohol carbonates such as, for example, dicaprylyl carbonate (Cetiol® CC), Guerbet carbonates based on fatty alcohols containing 6 to 18, preferably 8 to 10, carbon atoms, esters of benzoic acid with linear and/or branched C6 to C22 alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols and hydrocarbons or mixtures thereof.

In a preferred embodiment, the composition comprises at least one compound (I) and at least one oil component and optionally at least one aroma chemical (X).

In a preferred embodiment, the composition comprises at least one compound (I), or at least one compound (Ia), or at least one compound (Ib), or at least one compound (Ib′) or at least one compound (Ib″) and at least one oil component and optionally at least one aroma chemical (X).

### Antioxidants

It is to be understood that antioxidants are able to inhibit or prevent the undesired changes in the compositions to be protected caused by oxygen effects and other oxidative processes. The effect of the antioxidants consists in most cases in them acting as free-radical scavengers for the free radicals which arise during autoxidation.

In a preferred embodiment, the antioxidant is selected from the group consisting of
amino acids (for example glycine, alanine, arginine, serine, threonine, histidine, tyrosine, tryptophan) and derivatives thereof,
imidazoles (e.g. urocanic acid) and derivatives thereof,
peptides, such as D,L-carnosine, D-carnosine, L-carnosine (=β-Alanyl-L-histidine) and derivatives thereof,
carotenoids, carotenes (e.g. alpha-carotene, beta-carotene, lycopene, lutein) or derivatives thereof,
chlorogenic acid and derivatives thereof,
lipoic acid and derivatives thereof (for example dihydrolipoic acid),
auro-thioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof,
dilauryl thiodipropionate, distearyl thiodipropionate, thiodipropionic acid and derivatives thereof (esters, ethers, peptides, lipids, nucleotides, nucleosides and salts),
sulfoximine compounds (for example buthionine sulfoximines, homocysteine sulfoximine, buthionine sulfones, penta-, hexa-, heptathionine sulfoximine),
(metal) chelating agents (e.g. alpha-hydroxy fatty acids, palmitic acid, phytic acid, lactoferrin),
alpha-hydroxy acids (for example citric acid, lactic acid, malic acid),
humic acid, bile acid, bile extracts, bilirubin, biliverdin, boldin (=alkaloid from the plant Peumus boldus, boldo extract,
EDTA, EGTA and derivatives thereof,
unsaturated fatty acids and derivatives thereof (e.g. gamma-linolenic acid, linoleic acid, oleic acid),
folic acid and derivatives thereof,
ubiquinone and ubiquinol and derivatives thereof,
vitamin C and derivatives (for example ascorbyl palmitate, Mg ascorbyl phosphate, ascorbyl acetate),
tocopherols and derivatives (for example vitamin E acetate),
vitamin A and derivatives (for example vitamin A palmitate),
coniferyl benzoate of gum benzoin, rutic acid and derivatives thereof, alpha-glycosylrutin, ferulic acid, furfurylideneglucitol,
butylhydroxytoluene (BHT), butylhydroxyanisole (BHA),
nordihydroguaiacic acid, nordihydroguaiaretic acid, trihydroxybutyrophenone, uric acid and derivatives thereof, mannose and derivatives thereof,
superoxide dismutase,
zinc and derivatives thereof (for example ZnO, ZnSO4),
selenium and derivatives thereof (for example selenomethionine),
stilbenes and derivatives thereof (e.g. stilbene oxide, trans-stilbene oxide) and mixtures of two or more of the aforementioned.

In a preferred embodiment, the anti-oxidant is selected from the group consisting of pentaerythrityl, tetra-di-t-butyl-hydroxyhydrocinnamate, nordihydroguaiaretic acid, ferulic acid, resveratrol, propyl gallate, butylhydroxytoluene (BHT), butylhydroxyanisole (BHA), ascorbyl palmitate, tocopherol and mixtures of two or more of the aforementioned.

Preferably, the compositions according to the presently claimed invention can comprise the anti-oxidant in a total amount of 0.001 to 25 wt.-%, preferably 0.005 to 10 wt.-%, more preferably 0.01 to 8 wt.-%, yet more preferably 0.025 to 7 wt.-%, even more preferably 0.05 to 5 wt.-%, based on the total weight of the composition.

In a preferred embodiment, the composition comprises at least one compound (I) and at least one antioxidant and optionally at least one aroma chemical (X).

In a preferred embodiment, the composition comprises at least one compound (I), or at least one compound (Ia), or at least one compound (Ib), or at least one compound (Ib′) or at least one compound (Ib″) and at least one anti-oxidant and optionally at least one aroma chemical (X).

### Deodorant-Active Agents

Deodorizing compositions (deodorants and antiperspirants) counteract, mask or eliminate body odors. Body odors are formed through the action of skin bacteria on apocrine perspiration which results in the formation of unpleasant-smelling degradation products.

Preferably, the deodorant-active agent is selected from the groups consisting of anti-perspirants, esterase inhibitors, antibacterial agents and mixtures of two or more of the aforementioned.

Suitable antiperspirant is selected from the group consisting of salts of aluminum, zirconium or zinc. Examples are aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, for example with amino acids, such as glycine. Aluminum chlorohydrate, aluminum zirconium tetrachlorohydrate, aluminum zirconium pentachlorohydrate and complex compounds thereof are preferably used.

Preferably, the anti-perspirant is selected from the group consisting of aluminum chloride, aluminum chlorohydrate, aluminum dichlorohydrate, aluminum sesquichlorohydrate, aluminum hydroxyallantoinate, aluminum chloride tartrate, aluminum zirconium trichlorohydrate, aluminum zirconium tetrachlorohydrate aluminum zirconium pentachlorohydrate and mixtures of two or more of the aforementioned.

Where perspiration is present in the underarm region, extracellular enzymes-esterases, mainly proteases and/or lipases are formed by bacteria and split the esters present in the perspiration, releasing odors in the process. Suitable esterase inhibitors are for example trialkyl citrates, such as trimethyl citrate, tripropyl citrate, triisopropyl citrate, tributyl citrate and, in particular, triethyl citrate. Esterase inhibitors inhibit enzyme activity and thus reduce odor formation. The free acid is probably released by the cleavage of the citric acid ester and reduces the pH value of the skin to such an extent that the enzymes are inactivated by acylation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, zinc glycinate and mixtures of two or more of the aforementioned.

Preferably, the esterase inhibitor is selected from the group consisting of trimethyl citrate, tripropyl citrate, triisopropyl citrate, tributyl citrate triethyl citrate, lanosterol, cholesterol, campesterol, stigmasterol, sitosterol sulfate, sitosterol phosphate, glutaric acid, glutaric acid monoethyl ester, glutaric acid diethyl ester, adipic acid, adipic acid monoethyl ester, adipic acid diethyl ester, malonic acid, malonic acid diethyl ester, citric acid, malic acid, tartaric acid, tartaric acid diethyl ester zinc glycinate and mixtures of two or more of the aforementioned.

Preferably, the compositions according to the presently claimed invention comprise the the esterase inhibitor in a total amount in the range of 0.01 to 20 wt.-%, preferably 0.1 to 10 wt.-% and more particularly 0.5 to 5 wt.-%, based on the total weight of the composition.

The term “anti-bacterial agents” as used herein encompasses substances which have bactericidal and/or bacteriostatic properties. Typically these substances act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Preferably, the antibacterial agent is selected from the group consisting of chitosan, phenoxyethanol, 5-chloro-2-(2,4-dichlorophenoxy)-phenol, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides and mixtures of two or more of the aforementioned.

Preferably, the composition according to the presently claimed invention comprises the antibacterial agent(s) in a total amount in the range of 0.01 to 5 wt. % and preferably 0.1 to 2 wt.-%, based on the total weight of the composition.

In a preferred embodiment, the composition comprises at least one compound (I) and at least one deodorant active agent and optionally at least one aroma chemical (X).

In a preferred embodiment, the composition comprises at least one compound (I), or at least one compound (Ia), or at least one compound (Ib), or at least one compound (Ib′) or at least one compound (Ib″) and at least one deodorant active agent and optionally at least one aroma chemical (X).

### Surfactant

Preferably, the surfactant is selected from the group consisting of anionic, non-ionic, cationic, amphoteric, zwitterionic surfactant and a mixture of two or more of the aforementioned. In yet another preferred embodiment, the surfactant is an anionic surfactant.

The compositions according to the invention usually contain the surfactant(s), in a total amount of 0 to 40 wt. %, preferably 0 to 20 wt. %, more preferably 0.1 to 15 wt. %, and particularly 0.1 to 10 wt. %, based on the total weight of the composition. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolysates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution.

Zwitterionic surfactants are surface-active compounds which contain at least one quaternary ammonium group and at least one COO(—) or SO3(-) group in the molecule. Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate. The fatty acid amide derivative known under the CTFA name of Cocamidopropyl Betaine is particularly preferred.

Ampholytic surfactants are also suitable, particularly as co-surfactants. Ampholytic surfactants are surface-active compounds which, in addition to a C8 to C18 alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalk-ylaminopropionate, cocoacylaminoethyl aminopropionate and acyl sarcosine.

Anionic surfactants are characterized by a water-solubilizing anionic group such as, for example, a carboxylate, sulfate, sulfonate or phosphate group and a lipophilic group. Dermatologically safe anionic surfactants are known to the practitioner in large numbers from relevant textbooks and are commercially available. They are, in particular, alkyl sulfates in the form of their alkali metal, ammonium or alkanolammonium salts, alkylether sulfates, alkylether carboxylates, acyl isethionates, acyl sarcosinates, acyl taurines containing linear C12-C18 alkyl or acyl groups and sulfosuccinates and acyl glutamates in the form of their alkali metal or ammonium salts.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as, for example, the dialkyl ammonium methosulfates and methyl hydroxyalkyl dialkoyloxyalkyl ammonium methosulfates marketed under the name of Stepantexe and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolysates.

In a preferred embodiment, the composition comprises at least one compound of formula (I) and at least one surfactant and optionally at least one aroma chemical (X).

In a preferred embodiment, the composition comprises at least one compound (I), or at least one compound (Ia), or at least one compound (Ib), or at least one compound (Ib′) or at least one compound (Ib″) and at least one surfactant and optionally at least one aroma chemical (X).

Suitable compositions are for example perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles and oils, such as lamp oils or oils for massage.

Examples for fine fragrances are perfume extracts, Eau de Parfums, Eau de Toilettes, Eau de Colognes, Eau de Solide and Extrait Parfum.

Body care compositions include cosmetic compositions and products for oral and dental hygiene, and can be selected from after-shaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, after-shave creams and lotions, tanning creams and lotions, hair care products such as e.g. hairsprays, hair gels, setting hair lotions, hair conditioners, hair shampoo, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks and deodorant creams, products of decorative cosmetics such as e.g. eye-liners, eye-shadows, nail varnishes, make-ups, lipsticks and mascara, and products for oral and dental hygiene, such as toothpaste, dental floss, mouth wash, breath fresheners, dental foam, dental gels and dental strips.

Hygiene articles can be selected from joss sticks, insecticides, repellents, propellants, rust removers, perfumed freshening wipes, armpit pads, baby diapers, sanitary towels, toilet paper, cosmetic wipes, pocket tissues, dishwasher and deodorizer.

Cleaning compositions, such as e.g. cleaners for solid surfaces, can be selected from perfumed acidic, alkaline and neutral cleaners, such as e.g. floor cleaners, window cleaners, dishwashing compositions both for handwashing and machine washing use, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners.

Textile detergent compositions can be selected from liquid detergents, powder detergents, laundry pretreatments such as bleaches, soaking agents and stain removers, fabric softeners, washing soaps, washing tablets.

Food means a raw, cooked, or processed edible substance, ice, beverage or ingredient used or intended for use in whole or in part for human consumption, or chewing gum, gummies, jellies, and confectionaries.

A food supplement is a product intended for ingestion that contains a dietary ingredient intended to add further nutritional value to the diet. A dietary ingredient may be one, or any combination, of the following substances: a vitamin, a mineral, an herb or other botanical, an amino acid, a dietary substance for use by people to supplement the diet by increasing the total dietary intake, a concentrate, metabolite, constituent, or extract. Food supplements may be found in many forms such as tablets, capsules, soft gels, gel caps, liquids, or powders.

Pharmaceutical compositions comprise compositions which are intended for use in the diagnosis, cure, mitigation, treatment, or prevention of disease as well as articles (other than food) intended to affect the structure or any function of the body of man or other animals.

Crop protection compositions comprise compositions which are intended for the managing of plant diseases, weeds and other pests (both vertebrate and invertebrate) that damage agricultural crops and forestry.

Preferably, the composition according to the invention, further comprises at least one auxiliary agent selected from the group consisting of preservatives, abrasives, anti-acne agents, agents to combat skin aging, anti-cellulite agents, antidandruff agents, anti-inflammatory agents, irritation-preventing agents, irritation-alleviating agents, astringents, sweat-inhibiting agents, antiseptics, anti-statics, binders, buffers, carrier materials, chelating agents, cell stimulants, care agents, hair removal agents, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono- or polyunsaturated fatty acids, α-hydroxy acids, polyhydroxy fatty acids, liquefiers, dyes, color-protection agents, pigments, anti-corrosives, polyols, electrolytes and silicone derivatives.

One embodiment of the invention is directed to the method of preparing a composition comprising:
(i) at least one aroma compound (X)other than compounds according to the present invention or
(ii) at least one non-aroma chemical carrier, or
(iii) both of (i) and (ii).

For example, the method can be carried out by mixing the at least one compound of formula (I) and:
(i) at least one aroma compound(X) other than compounds for formula (I)
(ii) at least one non-aroma chemical carrier, or
(iii) both of (i) and (ii).

The invention is also directed to a method for boosting the aroma impression of a composition such as a fragranced composition, wherein the method comprises incorporating the compound of the presently claimed invention described herein into a composition.

In particular, the invention is directed to a method of preparing a perfume composition, body care composition, hygiene article, cleaning composition, textile detergent composition, composition for scent dispensers, food, food supplement, pharmaceutical composition or crop protection composition, comprising including the compound of the presently claimed invention described herein in a perfume composition, body care composition, hygiene article, cleaning composition, textile detergent composition, composition for scent dispensers, food, food supplement, pharmaceutical composition or crop protection composition.

In one embodiment the invention is directed to a method for imparting a note reminiscent of amber note, fruity note, citrus note, sweet note, woody note, herbal note, camphoraceous note, technical note and spicy note, elements to a perfume composition, body care composition, hygiene article, cleaning composition, textile detergent composition, composition for scent dispensers, food, food supplement, pharmaceutical composition or crop protection composition, which comprises including a compound of the presently claimed invention in a perfume composition, body care composition, hygiene article, cleaning composition, textile detergent composition, composition for scent dispensers, food, food supplement, pharmaceutical composition or crop protection composition.

### Amounts

Generally, the total amount of compounds of formula (I) in the compositions, methods and uses according to the present invention is typically adapted to the particular intended use or the intended application and can, thus, vary over a wide range. As a rule, the customary standard commercial amounts for aroma chemicals, preferably for scents are used.

Preferably the compositions according to the invention comprise compounds of formula (I) in a total amount of 0.001 to 99.9 wt. %, based on the total weight of the composition.

Particularly, the compositions comprise compounds of formula (I) in a total amount of 0.001 to 99.5 wt. %, preferably of 50 to 99 wt. %, more preferably of 80 to 95 wt. % and in particular of 90 to 95 wt. %, based on the total weight of the composition.

Particularly, the compositions comprises compounds of formula (I) in a total amount of 0.005 to 80 wt. %, preferably of 0.1 to 30 wt. %, more preferably of 1 to 20 wt. %, and in particular of 5 to 15 wt. %, based on the total weight of the composition.

Particularly, the compositions comprise compounds of formula (I) in a total amount of 0.001 to 20 wt. %, preferably of 0.005 to 6 wt. %, more preferably of 0.05 to 4 wt. %, and in particular of 0.1 to 3 wt. %, based on the total weight of the composition.

In each of the above embodiment, the compound of formula (I) can be replaced by compounds of formula (Ia), or compounds of formula (Ib), or compounds of formula (Ib′) or compounds of formula (Ib″).

### Process

### General Method:

The compounds of the present invention have been prepared via two different routes namely Route 1 and Route 2.

Both routes start from the corresponding dialkyl-pentenal (C).

Route 1 represents the synthesis of the dialkyl-pentadien-ether. This route involves the formation of acetal under acidic condition and azeotropic removement of water. Thereafter the acetal is cleaved at higher reaction temperatures to an E/Z-mixture of the 3,3-dialkyl-pentadien-ether. The formed dialkyl-pentadien-ether show a high chemical and thermal stability. Furthermore, the synthesized compounds have interesting fragrance profiles, which is described below in table 1.

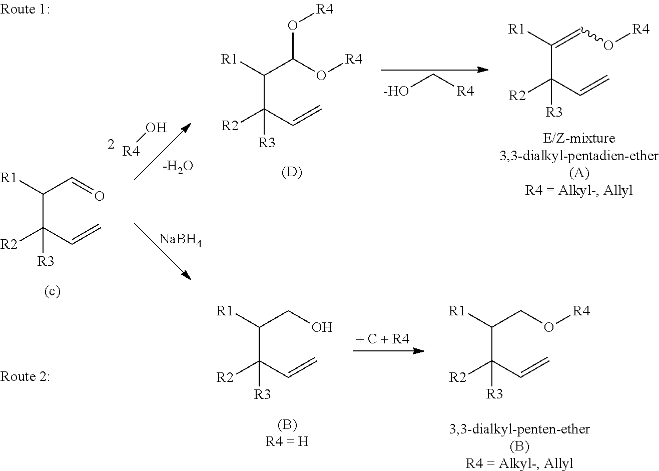

Route 2 was employed for the synthesis for the 3,3-dialkyl-penten-ether derivatives wherein the aldehyde is reduced to the corresponding alcohol. After deprotonation (e. g. NaH) the alcoholate is then alkylated in a Williamson ether synthesis to the corresponding product. The formed 3,3-dialkyl-penten-ether have a slightly different odor profile, compared to the 3,3-dialkyl-pentadien-ether, which is described below.

The aldehydes (C) for the synthesis of the 3,3-dialkyl-penten-ether & 3,3-dialkyl-pentadien-ether have been prepared from already available aldehydes (G) and allylic alcohols (H). In a first step, the acetal (F) is formed in the presence of a strong acid (e. g. p-TsOH) and azeotropic removement of water.

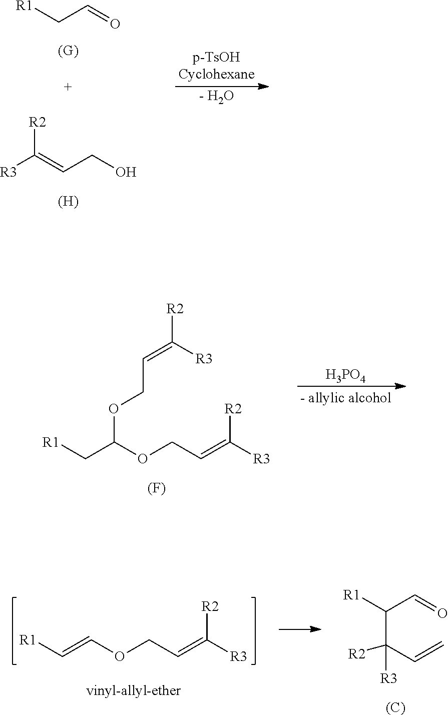

In a second step, the acetal (F) is cleaved to the vinyl-allyl-ether intermediate in the presence of a weaker acid (e. g. H₃PO₄). At the chosen reaction temperature of 140-150° C., the vinyl-allyl-ether intermediate was not isolated, but directly rearranged in a Claisen reaction to the 3,3-dialkyl-pentenal (C). The formed product (C) was removed from the reaction mixture via distillation, to prevent side reactions and decomposition of (C). The 3,3-dialkyl-pentenal was then further purified via distillation or column chromatography.

### Embodiments

In the following, there is provided a list of embodiments to further illustrate the present disclosure without intending to limit the disclosure to the specific embodiments listed below.
1. A compound of formula (I)

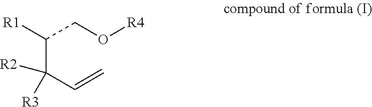

wherein formula (I) comprises,
the compounds of formula (Ia)

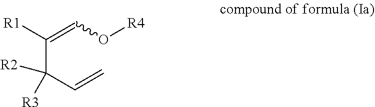

and compound of formula (Ib)

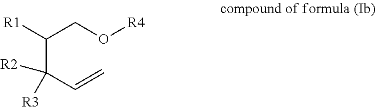

where
R₁ is linear or branched C₂-C₄ alkenyl or C₆-C₇ aryl,
R₂ is linear or branched C₁-C₄ alkyl,
R₃ is linear or branched C₁-C₄ alkyl,
R₄ is H, linear or branched C₁-C₅ alkyl, linear or branched C₂-C₅ alkenyl,
where the broken bond of compound of formula (I) is a single or double bond
or its salt or stereoisomer thereof.
2. The compound of formula (Ib) according to embodiment 1, wherein the formula (Ib) comprises the compounds of formula (Ib′)

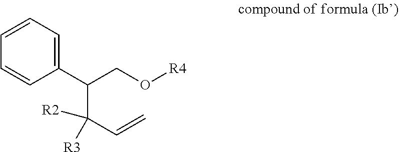

where
R₂ is linear or branched C₁-C₄ alkyl,
R₃ is linear or branched C₁-C₄ alkyl,
R₄ is H, linear or branched C₁-C₄ alkyl,
and the compounds of formula (Ib″)

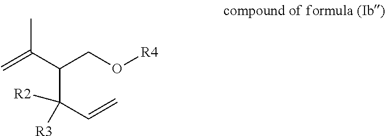

where
R₂ is linear or branched C₁-C₄ alkyl,
R₃ is linear or branched C₁-C₄ alkyl,
R₄ is linear or branched C₂-C₅ alkyl, linear or branched C₂-C₅ alkenyl.
3. The compound of formula (Ia) according to embodiment 1, wherein
   R₁ is linear or branched C₂-C₄ alkenyl,
   R₂ is methyl,
   R₃ is methyl,
   R₄ is linear C₁-C₃ alkyl.
4. The compound of formula (Ia) according to embodiment 1, wherein
   R₁ is linear or branched C₂-C₄ alkenyl,
   R₂ is methyl,
   R₃ is methyl,
   R₄ is branched C₅ alkenyl.
5. The compound of formula (Ib′) according to embodiment 2, wherein
   R₂ is methyl,
   R₃ is methyl,
   R₄ is H.
6. The compound of formula (Ib′) according to embodiment 2, wherein
   R₂ is methyl,
   R₃ is methyl,
   R₄ is linear C₁-C₃ alkyl.
7. The compound of formula (Ib″) according to embodiment 2, wherein
   R₂ is methyl,
   R₃ is methyl,
   R₄ is C₅ alkenyl.
8. The compound of formula (Ib″) according to embodiment 2, wherein
   R₂ is methyl,
   R₃ is methyl,
   R₄ is linear C₂-C₃ alkyl.
9. Use of at least one compound of formula (I) according to any of the embodiments 1 to 8 to impart an aroma impression to a composition.
10. Method of imparting an aroma impression to a composition comprising at least the step of adding at least one compound of formula (I) according to any of the embodiments 1 to 8 to a composition.
11. The use or method according to any of the embodiments 9 to 10, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.
12. The use or method according to any of the embodiments 9 to 10, wherein the aroma impression is selected from the group consisting of an amber note, fruity note, sweet note, floral note, musty note, animalic note, technical note and spicy note or any combination of two or more of these notes.
13. The use or method according to any of the embodiments 9 to 10, wherein compounds of formula (I) are used in a total amount in the range of ≥0.01 wt. % to ≤70.0 wt. %, based on the total weight of the composition.
14. The composition comprising at least one compound of formula (I) according to any of the embodiments 1 to 8 and,
   (i) at least one aroma chemical (X) other than the compound of formula(I), or
   (ii) at least one non-aroma chemical carrier, or
   (iii) both of (i) and (ii).
15. The composition according to embodiment 14, wherein the at least one compound of formula (I) is present in range of ≥0.01 wt. % to ≤70.0 wt. %, based on the total weight of the composition.
16. The composition according to embodiment 15, wherein the at least one compound of formula (I) is present in range of ≥1 wt. % to ≤70.0 wt. %, based on the total weight of the composition.
17. The composition according to embodiment 14, wherein the at least one non-aroma chemical carrier (ii) is selected from the group consisting of surfactants, oil components, antioxidants, deodorant-active agents and solvents.
18. The composition according to any of the embodiments 14 to 17, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.

### EXAMPLES

The present invention is illustrated in detail by non-restrictive working examples which follow. More particularly, the test methods specified hereinafter are part of the general disclosure of the application and are not restricted to the specific working examples.

### Analytical Method

The characterization is done by ¹³C-NMR and ¹H-NMR.

The ¹³C-NMR and ¹H-NMR spectra were measured on a Bruker DPX-500 spectrometer.

### GC Methods

### Method 1:

Agilent 6890A, column Agilent DB-WAX length 30 m, diameter: 0.32 mm, film 0.25 μm. T-program: Start 80° C., 4° C./min to 230° C., 4 min isotherm 230° C.

### Method 2:

Agilent 6890N, column Restek RTX-200 length 30 m, diameter: 0.25 mm, film 0.5 μm. T-program: 50° C. isotherm 4 min, 6° C./min to 280° C., 10 min isotherm 280° C.

### General Method

The new compounds have been prepared via two different routes namely Route 1 and Route 2.

Both routes start from the corresponding dialkyl-pentenal (C).

Route 1 represents the synthesis of the dialkyl-pentadien-ether. This route involves the formation of acetal under acidic condition and azeotropic removement of water. Thereafter the acetal is cleaved at higher reaction temperatures to an E/Z-mixture of the 3,3-dialkyl-pentadien-ether. The formed dialkyl-pentadien-ether show a high chemical and thermal stability. Furthermore, the synthesized compounds have interesting fragrance profiles, which is described below in table 1.

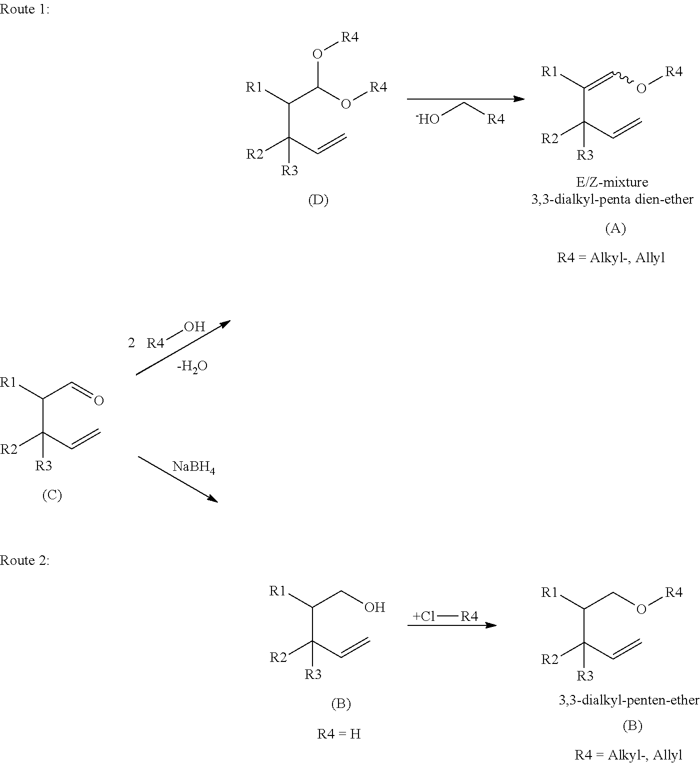

Route 2 was employed for the synthesis for the 3,3-dialkyl-penten-ether derivatives wherein the aldehyde is reduced to the corresponding alcohol. After deprotonation (e. g. NaH) the alcoholate is then alkylated in a Williamson ether synthesis to the corresponding product. The formed 3,3-dialkyl-penten-ether have a slightly different odor profile, compared to the 3,3-dialkyl-pentadien-ether, which is described below.

The aldehydes (C) for the synthesis of the 3,3-dialkyl-penten-ether & 3,3-dialkyl-pentadien-ether have been prepared from already available aldehydes (G) and allylic alcohols (H). In a first step, the acetal (F) is formed in the presence of a strong acid (e. g. p-TsOH) and azeotropic removement of water.

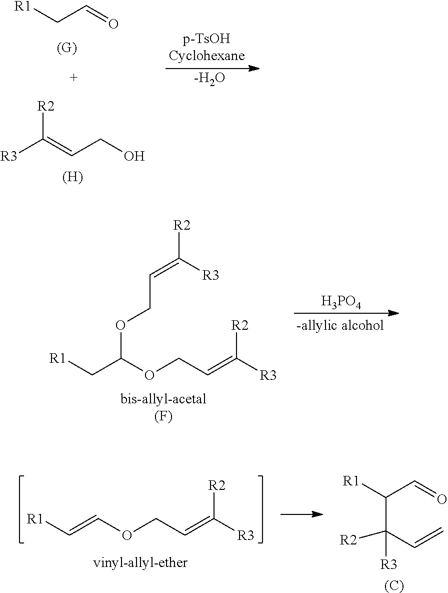

In a second step, the acetal (F) is cleaved to the vinyl-allyl-ether intermediate in the presence of a weaker acid (e. g. H₃PO₄). At the chosen reaction temperature of 140-150° C., the vinyl-allyl-ether intermediate was not isolated, but directly rearranged in a Claisen reaction to the 3,3-dialkyl-pentenal (C). The formed product (C) was removed from the reaction mixture via distillation, to prevent side reactions and decomposition of (C). The 3,3-dialkyl-pentenal was then further purified via distillation or column chromatography.

### Preparation of the Starting Materials (C):

### Example 1: Representative Procedure for the Synthesis of 2-isopropenyl-3,3-dimethyl-pent-4-enal

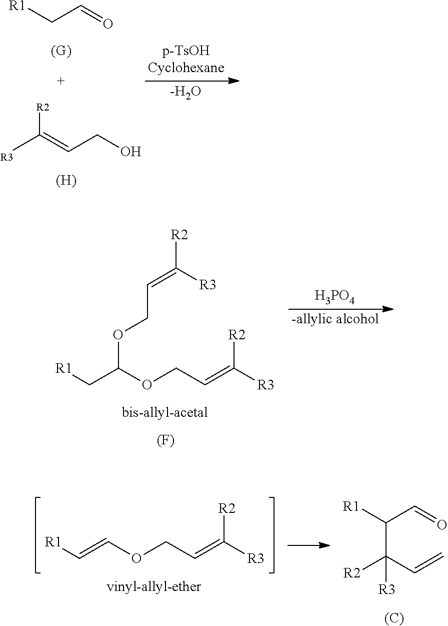

Prenal (400 g, 4.76 mol) and prenol (860 g, 10 mol) were dissolved in 1.5 l cyclohexane. After the addition of 250 ppm of p-TsOH the reaction mixture was refluxed (80-95° C.) and the formed water was removed via azeotropic distillation using a Dean-Stark apparatus. After refluxing for 3 h, 78.0 g water were removed and >90% of the Prenal was converted to the bis-allylacetal intermediate.

The reaction mixture was then neutralized with 180 ml NaHCO₃-solution (5%) and washed 3 times with 200 ml H₂O. The reaction mixture was then dried by azeotropic distillation and cyclohexane was distilled off at ambient pressure. The unreacted prenol and prenal were distilled at a pressure of 10-20 mbar and sump temperature of 80-90° C., to obtain 950 g of a pale-yellow residue in the sump. After GC-analysis (3% Prenol, 4.8% vinyl-allyl-ether-intermediate, 86% (F1), 1.4% high boiling compounds) the crude mixture was directly used for further reaction (acetal-cleavage). After the addition of 200 ppm of H₃PO₄ (85%), the acetal (F1) was cleaved at temperature of 140° C. The formed prenol and the product (C1) were removed continuously from the reaction mixture via distillation at a pressure of 5-10 mbar over an attached Vigreux column with a distillation bridge. The distillate stream (604 g, GC-analysis: 35% prenol, 47% (C1), 6.6% (F1)) was either directly used in the further transformations or purified via vacuum distillation.

For purification, 150 g of the distillate stream were transferred to a distillation apparatus, equipped with a spinning disk column, condenser, reflux controller and vacuum pump. The product was then purified in a fractional distillation with a sump temperature of 76-96° C., a pressure of 4 mbar and a reflux ratio of 5:1.

The analysis of the fractions was performed using GC:

| | |
|---|---|
| Fraction 1: 11.5 g | GC-analysis: 51.3% (C1) |
| Fraction 2: 9.4 g | GC-analysis: 73.2% (C1) |
| Fraction 3: 6.0 g | GC-analysis: 88.2% (C1) |
| Fraction 4: 10.3 g | GC-analysis: 95.6% (C1) |
| Fraction 5: 6.5 g | GC-analysis: 98.0% (C1) |
| Fraction 6: 16.3 g | GC-analysis: 97.6% (C1) |
| Fraction 7: 4.0 g | GC-analysis: 86.0% (C1) |
| Sump: 77 g | GC-analysis: 1.5% (C1), 37.2 |

The structure and purity of the product was verified with NMR-spectroscopy from fraction 5:

¹H NMR (500 MHZ, Chloroform-d): δ 1.15 (s, 6H), 1.6 (s, 3H), 2.65 (d, 1H), 4.8 (s, 1H), 4.95 (d, 1 H), 5.1 (d, 1 H), 5.95 (dd, 1 H), 9.7 (d, 1H).

¹³C NMR (125.77 MHz, CDCl₃): δ 202.08 d, 144.96 d, 139.55 s, 116.81 t, 112.66 t, 67.64 t, 39.44 s, 26.06 q, 25.46 q, 25.16 q.

### Example 2: Representative Procedure for the Synthesis of 3,3-dimethyl-2-phenyl-pent-4-enal (C2)

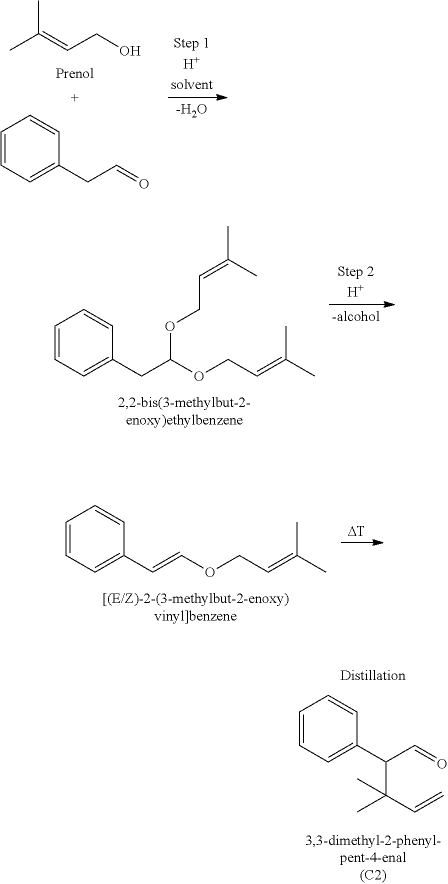

Phenylacetaldehyde (180 g, 1.5 mol) and prenol (258 g, 3.0 mol) were dissolved in 300 ml cyclohexane. After the addition of 250 ppm of p-TsOH the reaction mixture was refluxed (80-95° C.) and the water formed during the reaction was removed via azeotropic distillation using a Dean-Stark apparatus. After refluxing for 2 h, 25.5 g water were removed and >90% of the Phenylacetaldehyde was converted to 2,2-bis(3-methylbut-2-enoxy)ethylbenzene (=bis-allyl-acetal intermediate).

The reaction mixture was neutralized with 100 ml NaHCO₃-solution (5%) and washed 3 times with 50 ml H₂O. The reaction mixture was then dried by azeotropic distillation and cyclohexane was distilled of the reaction mixture to obtain 375.6 g of a pale-yellow residue. After GC-analysis (1.50% Prenol, 3.64% phenylacetaldehyde, 0.82% phenyl-dimethyl-pentenal, 0.45% dienylether, 88.03% bis-allyl-acetal) the crude mixture was directly used for further reaction (acetal-cleavage).

To the crude mixture, 100 ppm of H3PO4 (85%) was added and the apparatus was evacuated to 1-5 mbar and the temperature in the flask was gradually raised from 100° C. to 155° C. over a period of 3 h. As the reaction progressed, the bis-allyl-acetal was cleaved and the formed prenol was distilled from the reaction mixture. The unreacted prenol and phenylacetaldehyde were collected in a fraction 1 (67.9 g with 50% prenol, 40% phenylacetaldehyde). At a temperature of 130° C. (+/−5° C.) the rearrangement of [(E/Z)-2-(3-methylbut-2-enoxy)vinyl]benzene starts to occur and the distillate was collected in a fraction 2 (=267.5 g with 21% prenol, 71% 3,3-dimethyl-2-phenyl-pent-4-enal).

The reaction was stopped and after cooling to room temperature 26 g residue from the reaction flask were obtained.

The fraction 2 (267.5 g) was further purified in a second fractional batch distillation (20 cm column with glass rings) at a pressure of 70 mbar(fraction 1)−1 mbar (other fractions).

### Distillation 2:

Fr.1: Sump temp<140° C./T cond<50° C./70 mbar, 62.9 g, GC: 94.8% prenol
Fr.2: Sump temp=94-118° C./T cond=39-80° C./1 mbar, 14.9 g, GC: 57.9% phenylacetaldehyde.
Fr.3: Sump temp=118-119° C./Tcond=80-82° C./1 mbar, 25 g, GC: 97.0% (C2)
Fr.4: Sump temp=119-121° C./Tcond=81-82° C./1 mbar, 70 g, GC: 98.7% (C2)
Fr.5: Sump temp=121-127° C./Tcond=82° C./1 mbar, 53.6 g, GC: 98.0% (C2)
Fr.6: Sump temp=127° C./Tcond=82-79° C./1 mbar, 28 g, GC: 96.9% (C2) Sump+Cooling trap: 7.5 g+3 g

The product (3,3-dimethyl-2-phenyl-pent-4-enal (C2)) was obtained with a purity of >98% and was stabilized by the addition of tocopherol (100 ppm) to the fractions 3, 4, 5 & 6.

The structure and purity of the product was verified with NMR-spectroscopy:

¹H NMR (500 MHZ, Chloroform-d): δ 1.05 (s, 3H), 1.15 (s, 3H), 3.3 (s, 1H), 4.95 (d, 1H), 5.1 (d, 1H), 6.0 (dd, 1H), 7.2-7.4 (m, 5 Ar—H), 9.8 (s, —CHO)
¹³C NMR (125.77 MHz, CDCl₃): δ 202.07 d, 144.74 d, 134.65 s, 130.48 d, 130.48 d, 128.30 d, 128.30 d, 127.45 d, 113.22 t, 67.63 d, 40.05 s, 26.49 q, 24.80 q.
   Preparation of dialkylpentadien-ether Fragrances (A):

### Example 3: Representative Procedure for the Synthesis of (3Z/E-2,4,4-trimethyl-3-(3-methylbut-2-enoxymethylene)hexa-1,5-diene (A1)

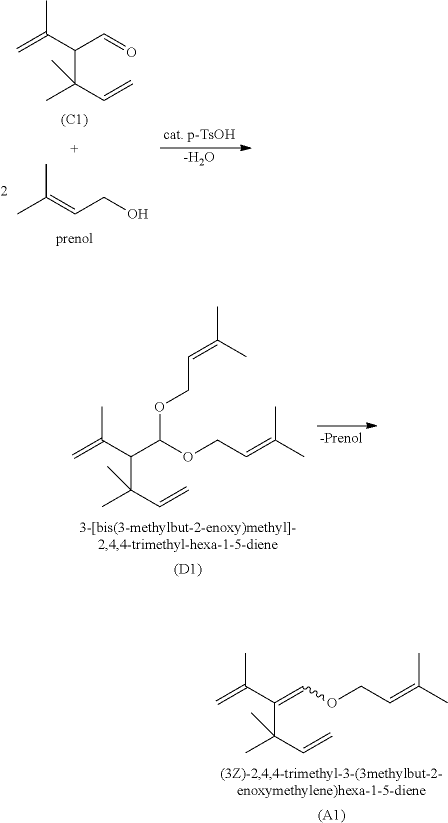

2-Isopropenyl-3,3-dimethyl-pent-4-enal (C1) (250 g, 0.8 mol) and prenol (200 g, 2.3 mol) were dissolved in 300 ml cyclohexane. After the addition of p-TsOH (0.05 g), the reaction mixture was refluxed (80-95° C.) and the water formed during the reaction was removed via azeotropic distillation using a Dean-Stark apparatus. After refluxing for 12 h, 11.8 g water were removed and >75% of the aldehyde (C1) was converted to the bis-allylacetale intermediate (D1). The reaction mixture was washed with 100 ml of a saturated NaHCO₃ solution. After phase separation, the organic layer was dried with Na₂SO₄, filtered and the cyclohexane was distilled off. 274.5 g of a pale red liquid product was obtained.

Analysis via GC showed the following composition of the residue: 0.8% Prenol, 8.6% Prenol-Ether, 6.1% (C1), 11.6% Citral (Z+E), 5.8% 3-methyl-1,1-bis(3-methylbut-2-enoxy)but-2-ene, 26.2% (D1) and 21% of high boiling compounds (e. g. (2E/Z)-3,7-dimethyl-4-(3-methylbut-2-enyl)octa-2,6-dienal).

For purification of (D1), the residue was distilled in batch distillation apparatus with a 15 cm packed column. At a vacuum of 1-2 mbar and a sump temperature of 150-160° C. a fraction (55.2 g) with a purity of 90% of the bis-allyl-acetal (D1) was isolated.

To the concentrated acetal (D1) a catalytic amount of 85% H₃PO₄ (500 ppm) was added and the acetal (D1) was distilled again. At a vacuum of 1-2 mbar and a sump temperature of 140-144° C. a fraction (19 g) of (3Z)-2,4,4-trimethyl-3-(3-methylbut-2-enoxymethylene)hexa-1,5-diene (A1) was obtained with a purity of 94%. The structure of the product (A1) was verified with NMR-spectroscopy:

¹H NMR (500 MHZ, Chloroform-d) of Z-isomer: δ 1.25 (s, 6 H), 1.65 (s, 3 H), 1.72 (s, 3 H), 1.75 (s, 3 H), 4.2 (d, 2 H), 4.55 (d, 1 H), 4.75 (d, 1 H), 4.85 (dd, 1 H), 4.95 (dd, 1 H), 5.45 (m, 1 H), 5.86 (s, 1 H), 6.1 (dd, 1 H).

¹³C NMR (125.77 MHz, CDCl₃) of Z-isomer: δ 147.84 d, 145.73 s, 141.92 d, 136.91 s, 128.37 s, 120.63 d, 113.96 t, 108.65 t, 68.57 t, 39.26 s, 26.82 q, 26.82 q, 25.74 q, 25.33 q, 18.11 q

¹H NMR (500 MHZ, Chloroform-d) of E-isomer: δ 1.2 (s, 6 H), 1.65 (s, 3 H), 1.7 (s, 3 H), 1.75 (s, 3 H), 4.25 (d, 2 H), 4.6 (d, 1 H), 4.9 (dd, 1 H), 5.05 (dd, 1 H), 5.4 (m, 1 H), 5.8 (dd, 1 H), 5.85 (s, 1 H).

¹³C NMR (125.77 MHz, CDCl₃) of E-isomer: δ 147.63 d, 142.26 s, 140.47 d, 137.04 s, 127.94 s, 120.63 d, 114.33 t, 110.33 t, 68.45 t, 39.59 s, 25.77 q, 26.52 q, 26.52 q, 24.99 q, 18.09 q.

Preparation of alkylpentadien-ether Fragrances (B):

### Example 4: Representative Procedure for the Synthesis of [1-(ethoxymethyl)-2,2-dimethyl-but-3-enyl]benzene (B5)

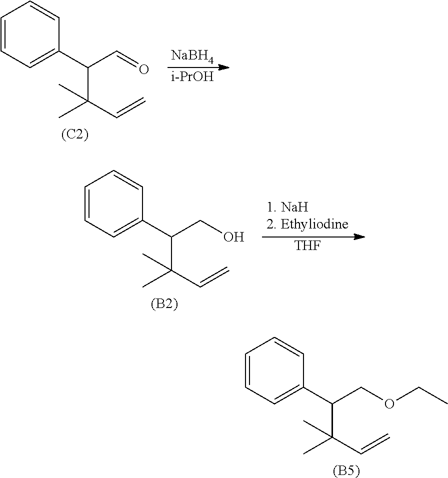

NaBH₄ (1.9 g, 50 mmol) was suspended in iso-propanol (25 ml) and cooled to 0-2° C. The 3,3-dimethyl-2-phenyl-pent-4-enal (C2) (18.2 g, 97 mmol) from the Claisen reaction was added within 0.5 h, further the reaction mixture was stirred for further 1.5 h at 0-2° C. The reaction was quenched with 25 ml of a saturated NH₄Cl-solution and methyl-tert-butyl-ether MTBE (50 ml) was added to extract the product into the organic layer. After phase separation the organic layer was dried with Na₂SO₄, filtrated and the solvent was removed in vaccum.

The product 3,3-dimethyl-2-phenyl-pent-4-en-1-ol (B2) was obtained as a crystalline solid (17.3 g) and a GC-purity of 99.2%.

For the formation of the ethyl-ether product (B5), NaH (160 mmol, 6.5 g) was suspended in dry THF (175 ml). The alcohol (B2), dissolved in 175 ml THF was added within 30 minutes at a temperature of 15-20° C. Afterwards ethyl iodide (4.43 g, 28.4 mmol) was added over a period of 15 minutes. Then the reaction mixture was stirred for 12 h at room temperature. The analysis of the reaction progress via gas chromatography indicated, that >95% of (B2) was converted to (B5). Therefore, the reaction mixture was quenched with H2O (10 ml) and the product was extracted with methyl-tert-butyl-ether (2×20 ml). After drying of the combined organic phases with Na₂SO₄, the solvent was removed in vacuo and the residue was purified via column chromatography (cyclohexane/ethylacetate 4:1). After distillation of the solvent, a colorless liquid (2.6 g) was obtained, which was analyzed via gas chromatography and NMR.
GC purity of (B5): 96.5%

The structure of the product (B5) was verified with NMR-spectroscopy:

¹H NMR (500 MHZ, Chloroform-d): δ 0.85 (s, 3H), 0.9 (s, 3H), 1.04 (t, 3H), 2.75 (m, 1H), 3.3 (m, 2 H), 3.75 (m, 2H), 4.9 (m, 2H), 5.65 (m, 1H), 7.18-7.3 (m, 5H).

¹³C NMR (125.77 MHz, CDCl₃): δ 146.76 d, 140.9 s, 129.52 d, 129.52 d, 127.51 d, 127.51 d, 126.16 d, 111.52 t, 71.26 t, 66.16 t, 55.21 d, 39.14 s, 27.28 q, 23.93 q, 15.03 q.

### Example 5: Representative Procedure for the Synthesis of 2-isopropenyl-3,3-dimethyl-pent-4-en-1-ol (B1)

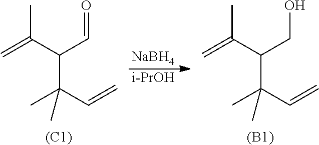

NaBH₄ (11.4 g, 0.3 mol) was suspended in iso-propanol (125 ml) and cooled to 0-2° C. The crude 2-isopropenyl-3,3-dimethyl-pent-4-enal (C1) (47%) (18.2 g, 97 mmol) from the Claisen reaction (s. Example 1) was added within 2.5 h. Afterwards the reaction mixture was stirred for further 2 h at 0-2° C. The reaction was quenched with 100 ml of a saturated NH₄Cl-solution and methyl-tert-butyl-ether MTBE (150 ml) was added to extract the product into the organic layer. After phase separation the organic layer was dried with Na₂SO₄, filtrated and the solvent was removed in vacuo. The yellowish oily residue (184 g with 47.9 area-% of the product B1) was purified via fractional batch distillation (20 cm column with random packings) at a pressure of 2 mbar.
Fr. 1: Sump temp<80° C./Tcond=61° C./2 mbar, 18.4 g, GC(area %): 23% B1
Fr.2: Sump temp=80-82° C./Tcond=61° C./2 mbar, 8.7 g, GC(area-%): 88% B1
Fr.3: Sump temp=82-84° C./Tcond=61° C./2 mbar, 17.8 g, GC(area-%): 92.1% B1
Fr.4: Sump temp=84-90° C./Tcond=61° C./2 mbar, 14.9 g, GC(area-%): 95.1% B1
Fr.5: Sump temp=90-100° C./Tcond=60° C./2 mbar, 11.9 g, GC(area-%): 94.9% B1
Fr.6: Sump temp=100-140° C./Tcond=55° C./2 mbar, 6.7 g, GC(area-%): 92.7% B1

The desired product 2-isopropenyl-3,3-dimethyl-pent-4-en-1-ol (B1) was obtained at a sump temperature of 84-100° C. and a head temperature von 58-60° C. In summary 26.8 g (Fr. 4+5) of B1 were obtained with a purity of 95%. The structure and purity of the product was confirmed with NMR-spectroscopy.

¹H NMR (500 MHz, Chloroform-d): δ δ 0.95 (s, 3 H), 1.05 (s, 3H), 1.17 (br, 1 OH), 1.75 (s, 3H), 2.2 (dd, 1H), 3.55 (dd, 1H), 3.6 (dd, 1H), 4.8 (d, 1H), 4.9-5 (m, 2 H), 5.1 (d, 1 H), 5.8-5.95 (dd, 1 H).

¹³C NMR (125.77 MHz, CDCl₃): 146.77 d, 144.44 s, 115.23 t, 111.36 t, 61.56 t, 58.72 d, 38.3 s, 26.96 q, 24.21 q, 23.38 q.

The combined fractions 4+5 were used for the olfactory evaluation of (B1)

### Example 6: Representative Procedure for the Synthesis of 2,4,4-trimethyl-3-(3-methylbut-2-enoxymethyl)hexa-1,5-diene (B3)

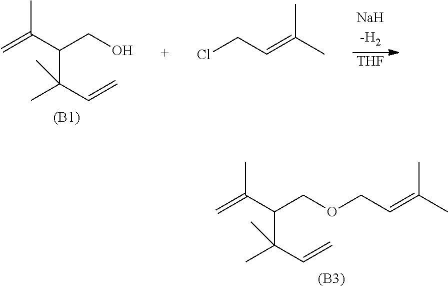

NaH 60 wt.-% in mineral oil (60 mmol, 2.4 g) was suspended in dry THF (40 ml). The 2-isopropenyl-3,3-dimethyl-pent-4-en-1-ol (B1, s. example 5) was dissolved in 5 ml dry THF, and was added within 30 minutes at a temperature of 15-20° C. The reaction mixture was stirred for 2 h at room temperature. To the reaction mixture, 1-chloro-3-methyl-2-butene (45 mmol, 4.7 g), dissolved in 5 ml dry THF, was added over a period of 15 minutes. The reaction mixture was heated to 50° C. and stirred for 6 h at 50° C. The analysis of the reaction progress via gas chromatography indicated, that >95% of (B1) was converted to (B3). The reaction mixture was then quenched with H2O (15 ml) and the product was extracted with methyl-tert-butyl-ether (2×25 ml). After drying of the combined organic phases with Na₂SO₄, the solvent was removed in vacuum and 9.3 g of a yellowish residue (87.6 GC area-% of (B3)) were obtained. The residue was purified via vacuum batch distillation (Claisen distillation bridge with cooler) at a pressure of 2 mbar. The sump temperature was steadily increased over a period of 3 h to 90° C. and fractions were collected. Fraction with different purities of 2,4,4-trimethyl-3-(3-methylbut-2-enoxymethyl)hexa-1,5-diene (B3) were obtained, which were analyzed with gas chromatography.
Fr.1: bp<78° C./2 mbar, 1.0 g, GC(area %): 72.61% (B3)
Fr.2: bp=78-82° C./2 mbar, 1.3 g, GC(area-%): 90.60% (B3)
Fr.3: bp=82-84° C./2 mbar, 2.2 g, GC(area-%): 95.9% (B3)
Fr.4: bp=84° C./2 mbar, 2.0 g, GC(area-%): 97% (B3)
Fr.5: bp=84° C./2 mbar, 1.1 g, GC(area-%): 97.2% (B3)

To confirm the structure of (B3), a sample of fraction 4 was analyzed with NMR-spectroscopy:

¹H NMR (500 MHZ, Chloroform-d): δ 0.95 (s, 3 H), 1.05 (s, 3H), 1.6 (s, 3 H), 1.70 (s, 3 H), 1.75 (s, 3 H), 2.25 (dd, 1 H), 3.3-3.6 (m, 2 H), 3.8-4.00 (m, 2 H), 4.8 (d, 1 H), 4.9-4.98 (m, 2 H), 5.0 (d, 1 H), 5.3-5.35 (m, 1 H), 5.8-5.95 (dd, 1 H).

¹³C NMR (125.77 MHz, CDCl₃): 147.14 d, 144.86 s, 136.38 s, 121.40 d, 114.21 t, 110.98 t, 69.66 t, 67.30 t, 55.52 d, 38.39 s, 27.21 q, 25.80 q, 24.44 q, 22.68 q, 18.01 q.

### Example 7: Representative Procedure for the Synthesis of 3-(ethoxy-methyl)-2,4,4-trimethyl-hexa-1,5-diene (B4)

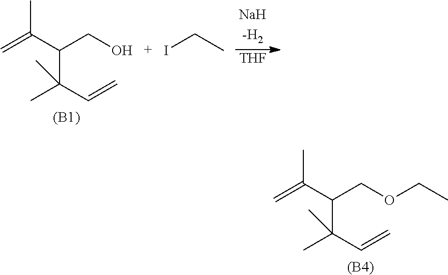

NaH 60 wt.-% in mineral oil (60 mmol, 2.4 g) was suspended in dry THF (40 ml). 2-isopropenyl-3,3-dimethyl-pent-4-en-1-ol (B1, s. example 5) (40 mmol, 6.16 g), was dissolved in 5 ml dry THF, and was added within 30 minutes at a temperature of 15-20° C. The reaction mixture was stirred for 2 h at room temperature. To the reaction mixture, ethyl iodine (45 mmol, 7.2 g), dissolved in 5 ml dry THF, was added, and the addition was carried out over a period of 15 minutes. Then the reaction mixture was heated to 50° C. and stirred for 2 h at 50° C. After the addition of further ethyl iodine (15.6 mmol, 2.5 g), the reaction mixture was stirred for additional 5 h at 50° C. The analysis of the reaction progress via gas chromatography indicated, that >95% of (B1) was converted to (B4). Further, the reaction mixture was quenched with an aqueous ammonia solution (15 ml) and the product was extracted with methyl-tert-butyl-ether (2×25 ml). The combined organic phases were washed with brine, dried with Na₂SO₄ and the solvent was removed in vacuo to obtain 6.9 g of a yellowish residue (89.4 GC area-% of B4).

The residue was purified via column chromatography at silica gel with cyclohexane (97.5 wt.-%) and ethyl acetate (2.5 wt.-%) as eluent. 3.6 g of 3-(ethoxy-methyl)-2,4,4-trimethyl-hexa-1,5-diene (B4) were obtained with a purity of 98.4%.

The structure and purity of the product was confirmed with NMR-spectroscopy.

¹H NMR (500 MHZ, Chloroform-d): δ 1.0 (s, 3H), 1.05 (s, 3H), 1.25 (t, 3H), 1.75 (s, 3H), 2.25 (dd, 1H), 3.4 (dd, 2H), 3.5 (dt, 2H), 4.75 (s, 1H), 4.8-4.95 (m, 2H), 5.0 (s, 1H), 5.75 (dd, 1H),

¹³C NMR (125.77 MHz, CDCl₃): 147.15 d, 144.95 s, 114.08 t, 111.01 t, 70.2 t, 66.2 t, 55.49 d, 38.39 s, 27.21 q, 24.41 q, 22.78 q, 15.11 q.

### Olfactory Impression

In order to test the quality and intensity of the compounds of the present invention, scent strip tests were performed.

For this purpose, strips of absorbent paper were dipped into a solution containing 1 to 10 wt. % of the compound to be tested in triethyl citrate. After evaporation of the solvent (about 30 s) the scent impression was olfactively evaluated by a trained perfumer.

Aroma impression of compounds of the present invention are indicated in the below table 1.

TABLE 1

| No. | Compound | Structure (with compound no.) | Olfactory impression |
|---|---|---|---|
| 1 | 2-isopropenyl-3,3- dimethyl-pent-4-en-1-ol | 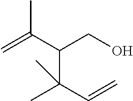 | Camphoraceous, citrus, herbal, woody |
| | | (B1) | |
| 2 | 3,3-dimethyl-2-phenyl- pent-4-en-1-ol | 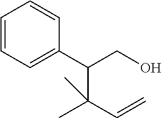 | Woody, Amber |
| | | (B2) | |
| 3 | 2,4,4-trimethyl-3-(3- methylbut-2- enoxymethyl)hexa-1,5- diene | 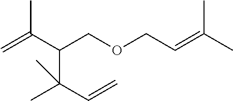 [SMILES: C=CC(C)(C)C(COCC=C(C)C)C(=C)C] | Technical (hot wax, hot iron, metallic) |
| | | (B3) | |
| 4 | 3-(ethoxymethyl)-2,4,4- trimethyl-hexa-1,5-diene | 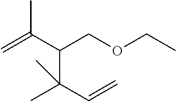 | Fruity, spicy |
| | | (B4) | |
| 5 | [1-(ethoxymethyl)-2,2- dimethyl-but-3- enyl]benzene | 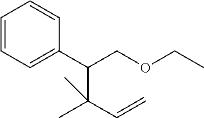 [SMILES: C=CC(C)(C)C(COCC)c1ccccc1] | Citrus, fruity, Gourmand (sweet) |
| | | (B5) | |
| 6 | (3Z/E)-2,4,4-trimethyl-3- (3-methylbut-2- enoxymethylene)hexa- 1,5-diene | 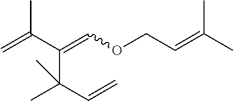 [SMILES: C=CC(C)(C)C(=COCC=C(C)C)C(=C)C] | -animalic (leather), floral, musty (dusty) |
| | | (A1) | |

### Advantageous Compositions

The compounds 1 to 6 indicated in table 1 were formulated as compositions according to tables 2 and 3. Compounds 1 to 6 from table 1 are labelled as “compound A” in table 2 and 3.

TABLE 2
Compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl)oxybutan-2-ol) | 4 | 4 |
| Ethyl-2-methyl butyrate | 4 | 4 |
| Geranyl acetate | 5 | 5 |
| Helional (3-(1,3-benzodioxol-5-yl)-2-methylpropanal) | 10 | 10 |
| Manzanate (ethyl 2-methylpentanoate) | 10 | 10 |
| Amberwood (ethoxymethoxycyclododecane) | 10 | 10 |
| Hexyl acetate | 11 | 11 |
| Benzyl salicylate | 12 | 12 |
| Magnolan (2,4-dimethyl-4,4a,5,9b-tetrahydroindeno[1,2- | 15 | 15 |
| d][1,3]dioxine) | | |
| Verdox (2-tert-butylcyclohexyl) acetate) | 25 | 25 |
| Bergamot oil bergaptene free | 25 | 25 |
| Linalol | 30 | 30 |
| Dipropylene glycol | 45 | 45 |
| Iso E Super (Tetramethyl acetyloctahydronaphthalenes) | 110 | 110 |
| Pyranol (4-methyl-2-(2-methylpropyl)oxan-4-ol) | 170 | 170 |
| Hedione (methyl 3-oxo-2-pentylcyclopentaneacetate) | 200 | 200 |
| Galaxolide 50% IPM (1,3,4,6,7,8-hexahydro-4,6,6,7,8,8- | 300 | 300 |
| hexamethylcyclopenta(g)-2-benzopyran 50% in isopropyl | | |
| myristate) | | |
| compound A | 20 | 50 |
| Total (part by weight) | 1020 | 1050 |

TABLE 3
Compositions 2A and 2B

| | 2A | 2B |
|---|---|---|
| Raspberry ketone (4-(4-hydroxyphenyl)butan-2-one) | 4 | 4 |
| Vanitrope (2-ethoxy-5-prop-1-enylphenol) | 6 | 6 |
| Cyclamen aldehyde (at least 90% 2-methyl-3-(p- | 10 | 10 |
| isopropylphenyl)propionaldehyde; secondary | | |
| component: 5% 3-(p-cumenyl)-2-methylpropionic acid) | | |
| Bicyclononalactone (3,4,4a,5,6,7,8,8a- | 10 | 10 |
| octahydrochromen-2-one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super (tetramethyl acetyloctahydronaphthalenes) | 20 | 20 |
| Sandela (3-[5,5,6-trimethylbicyclo[2.2.1]hept- | 30 | 30 |
| 2-yl]cyclohexan-1-ol) | | |
| Vanillin isobutyrate ((4-formyl-2-methoxyphenyl) 2- | 40 | 40 |
| methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4-dioxacycloheptadecane-5,17-dione) | 170 | 170 |
| Galaxolide 50% IPM (1,3,4,6,7,8-hexahydro-4,6,6,7,8,8- | 200 | 200 |
| hexamethylcyclopenta(g)-2-benzopyran 50% in isopropyl | | |
| myristate) | | |
| compound A | 20 | 50 |
| Total (part by weight) | 1020 | 1050 |

Composition according to table 2 and table 3 namely 1A, 1B, 2A, 2B could be included in various compositions selected from the group consisting of Deo pump spray, Clean hair-conditioner, Face wash gel, Foam bath concentrate, Hair gel, Self-foaming bodywash, Sprayable sun care emulsion, Sprayable sun protection emulsion, Emollient facial gel, 2-phases oil foam bath, Shampoos, Shower bath, Hydro-alcoholic AP/Deo pump spray, Aerosol, Aqueous/alcoholic AP/Deo roll-on, Styling Gel Type “Out of Bed”, Shaving Foam, Sensitive skin Baby shampoo, Body wash for Sensitive Skin, Gloss Enhancing Shampoo for Sensitive Scalp, Deo Stick, Baby Wipe, After shave balm, Face Gel, Face Day Care Cream, Face Cleanser, Body lotion, Sun Care SPF50+, Sprayable Lotion, Hand dish cleaner—regular, Hand dish cleaner—concentrate, Sanitary cleaner—concentrate, All-purpose cleaner, Anti-bacterial fabric softener, Detergent composition, Powder detergent composition and Liquid detergent composition.

A person skilled in art may be well versed with the various formulations for the above-mentioned products.

Compositions 1A, 1B, 2A and 2B can for example be formulated in specific formulations as disclosed in IP.com Number: IPCOM000258614D entitled New Aroma Chemicals pages 6 to 46, Table 1 to Table D13, wherein the “Fragrance Composition 1A” is replaced by identical amounts of compositions 1A, 1B, 2A or 2B.

1.-15. (canceled)

16. A compound of formula (I) 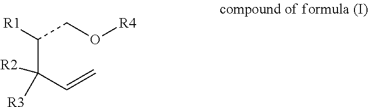
where the broken bond of compound of formula (I) is a single or double bond
wherein formula (I) comprises,
the compounds of formula (Ia) 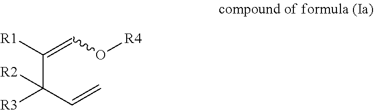
and the compounds of formula (Ib) 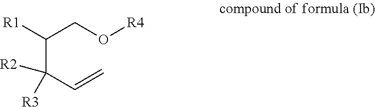
where
R₁ is linear or branched C₂-C₄ alkenyl or C₆-C₇ aryl
R₂ is linear or branched C₁-C₄ alkyl
R₃ is linear or branched C₁-C₄ alkyl
R₄ is H, linear or branched C₁-C₅ alkyl, linear or branched C₂-C₅ alkenyl
or its salt or stereoisomer thereof.

17. The compound of formula (Ib) according to claim 16, wherein the formula (Ib) comprises the compounds of formula (Ib′) 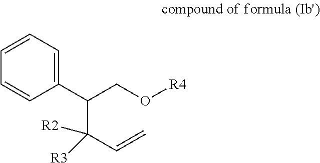
where
R₂ is linear or branched C₁-C₄ alkyl
R₃ is linear or branched C₁-C₄ alkyl
R₄ is H, linear or branched C₁-C₄ alkyl
and the compounds of formula (Ib″) 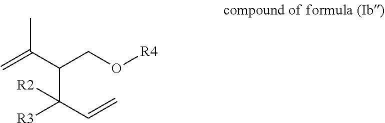
where
R₂ is linear or branched C₁-C₄ alkyl
R₃ is linear or branched C₁-C₄ alkyl
R₄ is linear or branched C₂-C₅ alkyl, linear or branched C₂-C₅ alkenyl.

18. The compound of formula (Ia) according to claim 16, wherein
R₁ is linear or branched C₂-C₄ alkenyl
R₂ is methyl
R₃ is methyl
R₄ is linear C₁-C₃ alkyl.

19. The compound of formula (Ia) according to claim 16, wherein
R₁ is linear or branched C₂-C₄ alkenyl
R₂ is methyl
R₃ is methyl
R₄ is branched C₅ alkenyl.

20. The compound of formula (Ib′) according to claim 17, wherein
R₂ is methyl
R₃ is methyl
R₄ is H.

21. The compound of formula (Ib′) according to claim 17, wherein
R₂ is methyl
R₃ is methyl
R₄ is linear C₁-C₃ alkyl.

22. The compound of formula (Ib″) according to claim 17, wherein
R₂ is methyl
R₃ is methyl
R₄ is C₅ alkenyl.

23. The compound of formula (Ib″) according to claim 17, wherein
R₂ is methyl
R₃ is methyl
R₄ is linear C₂-C₃ alkyl.

24. Use of at least one compound of formula (I) according to claim 16 to impart an aroma impression to a composition.

25. Method of imparting an aroma impression to a composition comprising at least the step of adding at least one compound of formula (I) according to claim 16 to a composition.

26. The use or method according to claim 24, wherein the aroma impression is selected from the group consisting of an amber note, fruity note, sweet note, floral note, musty note, animalic note, technical note and spicy note and any combination of two or more of these notes.

27. The use or method according to claim 24, wherein compounds of formula (I) are used in a total amount in the range of ≥0.01 wt. % to ≤70.0 wt. %, based on the total weight of the composition.

28. A composition comprising at least one compound of formula (I) according to claim 16 and,
(i) at least one aroma chemical (X) other than the compound of formula(I), or
(ii) at least one non-aroma chemical carrier, or
(iii) both of (i) and (ii).

29. The composition according to claim 28, wherein the at least one compound of formula (I) is present in range of ≥0.01 wt. % to ≤70.0 wt. %, based on the total weight of the composition.

30. The composition according to claim 28, wherein the at least one non-aroma chemical carrier (ii) is selected from the group consisting of surfactants, oil components, antioxidants, deodorant-active agents and solvents.
